SEQUENCE LISTING (2 sequences)

SEQ ID NO: 1 - protein, 74 aa, Bacillus stearothermophilus
MYTNSDFVVIKALEDGVNVIGLTRGADTRFHHSEKLDKGEVLIAQFTEHTSAIKVRGKAYIQTRHGVIESEGKK

SEQ ID NO: 2 - DNA, 225 bp, Bacillus stearothermophilus
atgtatacgaacagcgactttgttgtcattaaagcgcttgaagacggagtgaacgtcattggattgacgcgcggggcggatacacggttccatcactcggaaaagctcgataaaggcgaagtgttgatcgcccagtttacagagcacacgtcggcgattaaagtgagaggcaaggcgtatattcaaacgcgccatggcgtcattgagtcggaagggaaaaagtaa

(19) United States
(12) Patent Application Publication
(10) Pub. No.: US 2024/0139339 A1
(43) Pub. Date: May 2, 2024

(54) AN ARTIFICIAL PROTEIN-CAGE DECORATED WITH PARTICULAR MOLECULES ON THE EXTERIOR

(71) Applicant: UNIWERSYTET JAGIELLONSKI, Krakow (PL)

(72) Inventors: Jonathan HEDDLE, BIshop Auckland (GB); Yusuke AZUMA, Krakow (PL); Antonina NASKALSKA, Krakow (PL); Kinga BORZECKA-SOLARZ, Krakow (PL); Jan ROZYCKI, Krakow (PL); Szymon GAWEL, Wisniowa (PL)

(21) Appl. No.: 18/547,274

(22) PCT Filed: Feb. 24, 2022
(86) PCT No.: PCT/PL2022/050009

§ 371 Date: Oct. 10, 2023

(30) Foreign Application Priority Data
Feb. 24, 2021 (LU) LU102569
Feb. 24, 2021 (LU) LU102571
Feb. 24, 2021 (LU) LU102572
Feb. 24, 2021 (PL) P.437113
Feb. 24, 2021 (PL) P.437114
Feb. 24, 2021 (PL) P.437115

(51) Int. Cl. A61K 47/69; A61K 47/62

(52) U.S. Cl. CPC A61K 47/6949; A61K 47/62

(57) ABSTRACT

The present invention provides an artificial TRAP-cage decorated with particular molecules (proteins, peptides, small molecules, nucleic acids) on the exterior.

### FIELD OF THE INVENTION

The present invention falls within the biochemistry field. It is related to an artificial protein cage known as “TRAP-cage” decorated with particular molecules (proteins, peptides, small molecules, nucleic acids) on the exterior.

### BACKGROUND

Proteins that assemble into monodisperse cage-like structures are useful delivery/display vehicles for applications in biotechnology and medicine. Such protein cages exist in nature, e.g. viral capsids, but can also be designed and constructed in the laboratory.

A. D. Malay et al.: “Gold nanoparticle-induced formation of an artificial protein capsids”; Nano Letters, 12 (2012): 2056-2059 (which is hereby incorporated by reference) describes the TRAP-cage stabilised with gold atoms coordinating with TRAP rings, but disclosed TRAP-cage has no decorations, modifications or additional material on the exterior surface.

As such, inventors previously described that a cysteine-modified variant of the tryptophan RNA-binding attenuation protein from Geobacillus stearothermophilus, TRAP^{K35C, R64S}, can assemble into a hollow spherical structure composed of multiple ring-shape undecameric subunits via reaction with monovalent gold ions. The resulting protein cages exhibit an extremely high stability under many harsh conditions, but easily disassemble to the ring subunits in the presence of thiol- or phosphine-containing agents.

Based on this appealing platform, development of a general methodology to modify the cage exterior is essential to expand the utility of the artificial protein cages in drug delivery and vaccination.

The object of the invention is to provide chemical and enzymatic strategies to decorate the exterior surface of TRAP cage assemblies.

### SUMMARY OF THE INVENTION

The subject matter of the first aspect of invention is an artificial TRAP-cage comprising a selected number of TRAP rings and a plurality of external decorations attached, in particular covalently attached, thereto. Preferably, the artificial TRAP-cage comprises a selected number of TRAP rings which are held in place by cross-linkers. Preferably, the cross-linkers are molecular cross linkers or atomic metal cross linkers. Preferably the TRAP rings are cross-linked by gold.

Preferably, the external decorations are selected from the group comprising nanobodies, antibodies, epitopes, antigens, proteins, peptides, cell penetrating peptides, antigenic peptides, polypeptides, nucleic acids, signaling molecules, lipids, oligosaccharides, dye molecules, inorganic nanoparticles, specific ligands and small molecule therapeutics or fragments thereof.

The external decorations could also be antibody binding domains (preferably, variants Z15, Z34 and Z34c, all derived from Protein A, adhirons, anti-RBD domain of SARS-CoV-2 Spike protein. Preferably, the nanobodies are fluorescent protein (GFP)-nanobodies (a single-chain V_{H}H antibody domain developed with specific binding activity against GFP) or nanobodies (Nbs), an isolated, binding portion of an antibody. Preferably, the antibodies are antibodies targeting cell receptors or antibodies targeting cancer regulatory proteins such as anti-mutant p53 antibodies. Preferably, the proteins or peptides are receptor binding molecules, lectins, or transferrin, transferrin receptor binding proteins. They may be cytokines including chemokines interferons, interleukins Including interleukin-2 and artificial versions thereof, lymphokines, and tumour necrosis factors. They may be fluorescent proteins, preferably mCherry, tdTomato, dTomato. It may be albumin. Preferably, the peptides are peptide hormones, cell membrane disrupting peptides, T-cell-stimulating peptides or another type of peptides. Preferably, the nucleic acids are DNA, designed DNA nanostructures including those designed using the DNA origami technique, DNAzymes, RNA, mRNA, miRNA, siRNA, tRNA single stranded RNA, double stranded RNA, RNAzymes. Preferably, the nucleic acid is selected from the group comprising DNA, RNA, mRNA, siRNA, tRNA and micro-RNA. Preferably, the signaling molecules are steroid hormones, neurotransmitters, eicosanoids. Preferably, the lipids are phospholipids such as Phosphatidylcholine Preferably, the oligosaccharides are sucrose, fructose, or monosaccharides particularly glucose. Preferably, the dye molecules are fluorescent dyes. Preferably, the antigenic peptides are CpG dinucleotide motifs. Preferably, the inorganic nanoparticles are metal nanoparticles such as titanium oxide nanoparticles, iron, zinc, platinum, copper, sodium, cadmium, lanthanides, gadolinium, technetium, calcium, potassium, chromium, magnesium, molybdenum and salts or complexes thereof, or a carbon-based structure (e.g. a fullerene or a buckminsterfullerene, a single walled carbon nanotube or a multi-walled carbon nanotube).

Preferably, the external decoration is a viral, microbial or cancer antigen. Preferably the external decorations are the same or different from one another.

Preferably, at least one of the external decorations comprises a cell penetrating agent to promote intracellular delivery of the TRAP-cage.

Preferably, the cell penetrating agent is PTD4.

Preferably, the external surface of the TRAP-cage is modified to attach the external decoration by
(i) chemical modification;
(ii) enzymatic coupling;
(iii) bio-conjugation;
(iv) genetic coupling; and
(v) click chemistry.

Preferably, the external decoration is attached to an externally facing cysteine residue of the TRAP-cage. Preferably, the attachment is by chemical modification of the cysteine residue. Preferably, the chemical modification is by cysteine, maleimide-based conjugation.

Preferably, the chemical modification is via lysine amide-based conjugation.

Preferably, the attachment is by enzymatic coupling. Preferably, this is by sortase see e.g. Making and Breaking Peptide Bonds: Protein Engineering Using Sortase Maximilian Wei-Lin Popp, Prof. Dr. Hidde L. Ploegh Angew. Chem. Int. Ed. 22/2011 Volume 50, Issue 22 May 23, 2011 Pages 5024-5032, which is hereby incorporated by reference), Sfp (i.e. Phosphopantetheinyl transferases) (see e.g. Genetically encoded short peptide tag for versatile protein labeling by Sfp phosphopantetheinyl transferase PNAS Nov. 1, 2005 102 (44) 15815-15, which is hereby incorporated by reference) asparaginyl endoproteases, trypsin related enzymes or subtilisin-derived variants.

Addition of moieties, for example large macromolecules (proteins), peptides and small molecules (fluorescent dyes), to the exterior of TRAP-cage is described here using an enzymatic system this allows the coverage to be tuned. This is particularly advantageous because a 24 ring TRAP-cage, as described herein, comprises 264 identical monomers prior to modification. Thus, techniques which result in a one macromolecule bound per monomer would be sterically unfavourable. The enzymatic system described herein overcome this problem.

Preferably, the enzymatic coupling is via a peptide ligase. Preferably, the peptide ligase is selected from the group comprising sortases.

Preferably, the attachment is by bio-conjugation, preferably maleimide labelled fluorescent dyes for attachment of surface thiols (see e.g. Efficient Site-Specific Labeling of Proteins via Cysteines, Younggyu Kim, Sam O. Ho, Natalie R. Gassman, You Korlann, Elizabeth V. Landorf, Frank R. Collart, and Shimon Weiss, Bioconjugate Chemistry 2008 19 (3), 786-791, which is hereby incorporated by reference).

Preferably, the bio-conjugation is via an azide-reactive side chain. Preferably the azide-reactive side chain is DBCO.

Preferably, the attachment is by genetic coupling, whereby genetic material (e.g. a DNA sequence encoding a peptide or protein) is added after the sequence encoding the C-terminal region of TRAP protein with the result that the peptide or protein encoded by the sequence is located on the exterior of the TRAP-cage after the genetically coupled protein is expressed and purified and the cage assembled.

Preferably, the genetic coupling is via fusion to a C-terminus of TRAP.

Preferably, the external decoration is conjugated using SpyCatcher/SpyTag conjugation, preferably to an exterior surface of the TRAP-cage. Preferably, the SpyCatcher/SpyTag conjugation of the guest cargo to an exterior surface of the TRAP-cage. Preferably, the SpyCatcher is introduced in a region of TRAP rings which faces to the exterior when assembled into TRAP-cages. The external decorations will comprise a SpyTag. The Spy Tag may be fused to the C-terminus of TRAP protein.

Preferably, the N-terminus of the decoration to be attached to the TRAP-cage is fused to a C-terminus sequence of TRAP that is available on the exterior of the TRAP-cage.

Preferably, the TRAP-cage according to the invention further includes an internal or guest cargo encapsulated therein.

Preferably, the cargo is a protein, preferably selected from the group comprising an enzyme (e.g. protease, a nuclease, hydrogenase, dehydrogenase, lipase, lyase, ligase, transferase, reductase, recombinase, nuclease acid modification enzyme. or other type of enzyme) an antigen, an antibody. Or the cargo is another type of protein biological macromolecule (e.g. a sterol, steroid or a fatty acid). Or the cargo is a lipid, a peptide (e.g. a peptide hormone, a cell membrane disrupting peptide, a T-cell-stimulating peptide or another type of peptides) a nucleic acid (e.g. DNA, designed DNA nanostructures including those designed using the DNA origami technique, DNAzymes, RNA, mRNA, miRNA, siRNA, tRNA single stranded RNA, double stranded RNA, RNAzymes), a small molecular cargo such as a drug, a peptide nucleic acids (PNA), a carbon-based structure (e.g. a fullerene or a buckminsterfullerene, a single walled carbon nanotube or a multi-walled carbon nanotube) a metal (e.g. iron, zinc, platinum, copper, sodium, cadmium, lanthanides, gadolinium, technetium, calcium, potassium, chromium, magnesium, molybdenum and salts or complexes thereof), a toxin (e.g. a ligand targeted toxin, a protease activated toxin, pore forming/membrane disrupting peptides such as melittin and a toxin-based suicide gene therapeutic) or a nanoparticle (e.g. a metal nanoparticle such as gold, iron, silver, cobalt cadmium selenide, titanium oxide) or a core-shell metal nanoparticle such as a CdS/ZnS, CdSe/ZnS, CdSe/CdS, and InAs/CdSe nanoparticle. Preferably, the nucleic acid is selected from the group comprising DNA, RNA, mRNA, siRNA, tRNA and micro-RNA. Preferably, the therapeutic agent is an enzyme associated with an over-expression in a metabolic disorder or disease or an under expression in a metabolic disorder or disease. Preferably, the enzyme is selected from the group comprising hydrogenase, dehydrogenase, lipase, lyase, ligase, protease, transferase, reductase, recombinase and nuclease acid modification enzyme. Preferably, the therapeutic agent is selected from the group comprising a cancer therapeutic, an anti-infection therapeutic, a vascular disease therapeutic, an immune therapeutic, senolytic and a neurological therapeutic. Preferably, the metal is selected from the group comprising iron, zinc, platinum, copper, sodium, cadmium, lanthanide, gadolinium, technetium, calcium, potassium, chromium, magnesium, molybdenum and salts or complexes thereof. Preferably, the toxin is selected from the group comprising a ligand targeted toxin, a protease activated toxin, melittin and a toxin-based suicide gene therapeutic.

Preferably, the guest cargo is a protein. Preferably a fluorescent protein. Preferably GFP, mCherry or mOrange. Preferably interleukin-2 (IL-2) or Neoleukin-2/15 (NL-2).

Preferably, wherein the number of TRAP rings in the TRAP-cage is between 6 and 60, preferably between 7 and 55, preferably between 8 and 50, preferably between 9 and 45, preferably between 10 and 40, preferably between 11 and 35, preferably between 12 and 34, preferably between 13 and 33, preferably between 14 and 32, preferably between 15 and 31, preferably between 16 and 30, preferably between 17 and 29, preferably between 18 and 28, preferably between 19 and 27, preferably between 20 and 26. Preferably the number of TRAP rings in the TRAP-cage is less than 40, preferably less than 35, preferably less than 30. Preferably the number of TRAP rings in the TRAP-cage is more than 6, preferably more than 10, preferably more than 15, preferably more than 20.

Preferably, the number of TRAP rings in the TRAP-cage is between 12 and 24.

Preferably, the number of TRAP rings in the TRAP-cage is about 24, preferably 24. Preferably, the number of TRAP rings in the TRAP-cage is about 12, preferably 12. Preferably, the number of TRAP rings in the TRAP-cage is about 20, preferably 20. Preferably, the TRAP-cage according to the invention further includes an internal cargo encapsulated therein.

Preferably, opening of the cage is programmable. Preferably, said specific conditions corresponds to the specific cleavage characteristic of the cross-linker.

Preferably, the programmable opening of the cage is dependent on selection of a molecular or atomic metallic cross-linkers which hold the TRAP-rings in place in the TRAP-cage.

Preferably, the specific cleavage characteristic of the molecular cross-linker is selected from the group comprising:
(i) a reduction resistant/insensitive molecular cross-linker, whereby the cage remains closed under reducing conditions;
(ii) a reduction responsive/sensitive molecular cross-linker, whereby the cage opens under reducing conditions; and
(iii) a photoactivatable molecular cross-linker whereby the cage opens upon exposure to light.

Preferably, the reduction resistant/insensitive molecular cross-linker can be selected from the group comprising: bismaleimideohexane (BMH) and bis-bromoxylenes. Preferably, the reduction responsive/sensitive molecular cross-linker can be selected from the group comprising: dithiobismaleimideoethane (DTME). Preferably, the photoactivatable molecular cross-linker can be selected from the group comprising: bis-halomethyl benzene and its derivatives including 1,2-bis-bromomethyl-3-nitrobenzene (o-BBN), 2,4-bis-bromomethyl-1-nitrobenzene (m-BBN) and 1,3-bis-bromomethyl-4,6-dinitro-benzene (BDNB).

Preferably, the molecular cross-linker is a homobisfunctional molecular moiety and its derivatives. Preferably, homobisfunctional molecular cross-linker is bismaleimideohexane (BMH).

Preferably, the cage is resistant/insensitive to reducing conditions. Preferably the homobisfunctional molecular cross-linker is dithiobismaleimideoethane (DTME).

Preferably, the cage is responsive/sensitive to reducing conditions. Preferably the molecular cross-linker is a bis-halomethyl benzene and its derivatives.

Preferably, the molecular cross-linker is selected from the group comprising, 1, 2-bis-bromomethyl-3-nitrobenzene (BBN), bis-bromoxylene and 1,3-bis-bromomethyl-4,6-dinitro-benzene (BDNB).

Preferably, the molecular cross-linker is photolabile by exposure to UV light.

Preferably, the cage according to the invention comprises a mixture of different programmable molecular cross-linkers.

Preferably, the TRAP rings are variants.

Preferably, the artificial TRAP-cage protein is modified to comprise any one or more of the following mutations selected from the group comprising K35C, E48Q, E48K R64S, K35C/E48Q, K35C/E48K, and K35C/R64S. Preferably the artificial TRAP-cage protein is modified to comprise a K35C mutation. Preferably the artificial TRAP-cage protein is modified to comprise a K35C mutation or a K35C/E48Q mutation or a K35C/E48K mutation.

Preferably, the artificial TRAP-cage protein is modified to comprise any one or more of the following mutations selected from the group comprising K35C, K35H, R64S, K35C/R64S, K35H/R64S, S33C, S33H, S33C/R64S, S33H/R64S, S33C/K35H S33H/K35H, S33C/K35C, S33H/K35C.

Preferably, the TRAP-cages are stable in elevated temperatures, i.e. when the temperatures are elevated above normal room or human/animal body temperatures, preferably stable between 0 and 100° C., preferably stable between 15 and 100° C., preferably stable between 15 and 79° C., preferably stable up to 95° C., preferably stable at 95° C. and below.

Preferably, the TRAP-cages are stable in a non-neutral pH, preferably stable above pH 7 and below pH 7, preferably stable between pH 3 to 11, preferably stable between pH 4 to 10, preferably stable between pH 5 to 9.

Preferably, the TRAP-cages are stable in chaotropic agents (agents which disrupt hydrogen bonding in solution, which would disrupt or denature protein or macromolecular structures) or surfactants that would otherwise be expected to disrupt or denature protein or macromolecular structures. Preferably the cages show stability in n-butanol, ethanol, guanidinium chloride, lithium perchlorate, lithium acetate, magnesium chloride, phenol, 2-propanol, sodium dodecyl sulfate, thiourea, and urea. Preferably, the TRAP-cages are stable in up to 4 M GndHCl. Preferably, the TRAP-cages are stable in up to at least 7 M urea. Preferably, the TRAP-cages are stable in up to 15% of SDS. The stability of the cages described herein can be tested in standard conditions which would be known to the person of skill in the art using these agents to demonstrate said stability.

The cages described herein display unexpected stability in these conditions, providing more stable TRAP-cages than previously demonstrated.

The subject matter of the invention is also use of the cage according to the invention, as defined above, in delivery of a cargo or an external decoration attached to said cage in a controlled period and to a desired location.

The subject matter of the invention is also use of the artificial TRAP-cage according to the invention as a delivery vehicle for delivery of its external decoration.

Preferably, the delivery is for intracellular delivery. Preferably the delivery is for extracellular delivery.

The subject matter of the invention is also use of the artificial TRAP-cage according to the invention as a vaccine.

The subject matter of the invention is also use of the artificial TRAP-cage according to the invention for the treatment of an illness or disease condition selected from the group comprising cancer, vascular disease, cardiovascular disease, diabetes, infection, auto-immune condition, neurological/neurodegenerative disease, arthritis and respiratory disease.

The subject matter of a further aspect of the invention is also a method of making an artificial TRAP-cage, the method comprising:
(i) obtaining TRAP ring units by expression of the TRAP ring units in a suitable expression system and purification of the said units from the expression system;
(ii) conjugation of the TRAP ring units via at least one free thiol linkage with a cross-linker;
(iii) formation of the TRAP-cage by self-assembly and modification of an external surface of the formed TRAP-cage to what is appropriate for the external decoration that is to be attached to the cage exterior surface;
(iv) decorating the external surface of the TRAP-cage with an external decoration, preferably a moiety selected from the group comprising nanobodies, antibodies, epitopes, antigens, proteins, peptides, cell penetrating peptides, antigenic peptides, polypeptides, nucleic acids, signaling molecules, lipids, oligosaccharides, dye molecules, inorganic nanoparticles, specific ligands and small molecule therapeutics or fragments thereof; and
(v) purification and isolation of the TRAP-cages.

Preferably, the expression system in step (i) is selected from a cell-based expression system or other expression systems such as cell-free or plant expression systems.

Preferably, purification of the said units from the expression system of step (i) by using FPLC-based purification employing appropriate columns such as a mixture a of affinity based and size exclusion columns.

Preferably, step (ii) first comprises conjugation of the TRAP ring units via at least one metal cross-linker, preferably an atomic metal cross-linker. Step (ii) then comprises replacing the metal cross-linker with a molecular cross-linker. A molecular cross-linker may exchange metal atoms without changing orientation of the rings in the cage. Preferably, the metal is gold. This altered step (ii) preferably applies when the cross-linker is a photocleavable linkers, preferably wherein the cross linker is bromoxylene or bisbromobimane.

Preferably, the modification of step (iii) is selected from the group comprising:
(i) chemical modification;
(ii) enzymatic coupling;
(iii) bio-conjugation;
(iv) genetic coupling; and
(v) click chemistry.

Preferably, the external decoration is attached to an externally facing cysteine residue of the TRAP-cage. Preferably, the attachment is by chemical modification of the cysteine residue. Preferably, the chemical modification is by cysteine, maleimide-based conjugation.

Preferably, the chemical modification is via lysine amide-based conjugation.

Preferably, the attachment is by enzymatic coupling. Preferably, this is by sortase see e.g. Making and Breaking Peptide Bonds: Protein Engineering Using Sortase Maximilian Wei-Lin Popp, Prof. Dr. Hidde L. Ploegh, Angew. Chem. Int. Ed. 22/2011 Volume 50, Issue 22 May 23, 2011 Pages 5024-5032, which is hereby incorporated by reference), Sfp (i.e. Phosphopantetheinyl transferases) (see e.g. Genetically encoded short peptide tag for versatile protein labeling by Sfp phosphopantetheinyl transferase PNAS Nov. 1, 2005 102 (44) 15815-15, which is hereby incorporated by reference) asparaginyl endoproteases, trypsin related enzymes or subtilisin-derived variants.

Addition of large macromolecules to the exterior of TRAP-cage is described here using an enzymatic system this allows the coverage to be tuned. This is particularly advantageous because a 24 ring TRAP-cage, as described herein, comprises 264 identical monomers prior to modification. Thus, techniques which result in one external macromolecule bound per monomer could be sterically unfavourable. The enzymatic system described herein overcomes this problem wherein parameters of the enzymatic reaction can be modulated to achieve desired density of surface macromolecules decoration.

Preferably, the enzymatic coupling is via a peptide ligase. Preferably, the peptide ligase is selected from the group comprising sortases.

Preferably, the attachment is by bio-conjugation, preferably maleimide labelled fluorescent dyes for attachment of surface thiols (see e.g. Efficient Site-Specific Labeling of Proteins via Cysteines, Younggyu Kim, Sam O. Ho, Natalie R. Gassman, You Korlann, Elizabeth V. Landorf, Frank R. Collart, and Shimon Weiss, Bioconjugate Chemistry 2008 19 (3), 786-791, which is hereby incorporated by reference).

Preferably, the bio-conjugation is via an azide-reactive side chain. Preferably the azide-reactive side chain is DBCO.

Preferably the attachment is by genetic coupling or genetic fusion, whereby genetic material (e.g. a DNA sequence encoding a peptide or protein) is added after the sequence encoding the C-terminal region of TRAP protein, with the result that the peptide or protein encoded by the sequence is located on the exterior of the TRAP-cage after the genetically coupled protein is expressed and purified and the cage assembled.

Preferably, the genetic coupling is via fusion to a C-terminus of TRAP.

Preferably, the N-terminus sequence of the external decoration is fused to a C-terminus sequence of a TRAP protein on the exterior of the TRAP-cage.

Preferably, genetic fusion can comprise SpyCatcher/SpyTag conjugation of the external decoration to an exterior surface of the TRAP-cage. Preferably, the guest cargo is conjugated using SpyCatcher/SpyTag conjugation, preferably to an exterior surface of the TRAP-cage. Preferably, the SpyCatcher/SpyTag conjugation of the guest cargo to an exterior surface of the TRAP-cage. Preferably, the SpyCatcher is introduced in a region of TRAP rings which faces to the exterior when assembled into TRAP-cages. Here, the external decorations will comprise a SpyTag. The Spy Tag may be fused to the C-terminus of TRAP protein.

Preferably, the N-terminus of the molecule to be attached to the TRAP-cage is fused to a C-terminus sequence of TRAP that is available on the exterior of the TRAP-cage.

The external decorations could also be antibody binding domains (preferably, variants Z15, Z34 and Z34c, all derived from Protein A, adhirons, anti-RBD domain of SARS-CoV-2 Spike protein. Preferably, the nanobodies are fluorescent protein (GFP)-nanobodies (a single-chain V_{H}H antibody domain developed with specific binding activity against GFP) or nanobodies (Nbs), an isolated, binding portion of an antibody. Preferably, the antibodies are antibodies targeting cell receptors or antibodies targeting cancer regulatory proteins such as anti-mutant p53 antibodies. Preferably, the proteins are receptor binding molecules, lectins, or transferring, transferrin receptor binding proteins. They may be fluorescent proteins, preferably mCherry, tdTomato, dTomato. Preferably, the peptides are peptide hormones, cell membrane disrupting peptides, T-cell-stimulating peptides or another type of peptides. Preferably, the nucleic acids are DNA, designed DNA nanostructures including those designed using the DNA origami technique, DNAzymes, RNA, mRNA, miRNA, siRNA, tRNA single stranded RNA, double stranded RNA, RNAzymes. Preferably, the nucleic acid is selected from the group comprising DNA, RNA, mRNA, siRNA, tRNA and micro-RNA. Preferably, the signaling molecules are steroid hormones, neurotransmitters, eicosanoids. Preferably, the lipids are phospholipids such as Phosphatidylcholine Preferably, the oligosaccharides are sucrose, fructose, or monosaccharides particularly glucose. Preferably, the dye molecules are fluorescent dyes. Preferably, the antigenic peptides are CpG dinucleotide motifs. Preferably, the inorganic nanoparticles are metal nanoparticles such as titanium oxide nanoparticles, iron, zinc, platinum, copper, sodium, cadmium, lanthanides, gadolinium, technetium, calcium, potassium, chromium, magnesium, molybdenum and salts or complexes thereof, or a carbon-based structure (e.g. a fullerene or a buckminsterfullerene, a single walled carbon nanotube or a multi-walled carbon nanotube).

Preferably, the TRAP cage also comprises or holds an internal or guest cargo, preferably the cargo is a protein, preferably selected from the group comprising an enzyme (e.g. protease, a nuclease, hydrogenase, dehydrogenase, lipase, lyase, ligase, transferase, reductase, recombinase, nuclease acid modification enzyme. or other type of enzyme) an antigen, an antibody. Or the cargo is another type of protein biological macromolecule (e.g. a sterol, steroid or a fatty acid). Or the cargo is a lipid, a peptide (e.g. a peptide hormone, a cell membrane disrupting peptide, a T-cell-stimulating peptide or another type of peptides) a nucleic acid (e.g. DNA, designed DNA nanostructures including those designed using the DNA origami technique, DNAzymes, RNA, mRNA, miRNA, siRNA, tRNA single stranded RNA, double stranded RNA, RNAzymes), a small molecular cargo such as a drug, a peptide nucleic acids (PNA), a carbon-based structure (e.g. a fullerene or a buckminsterfullerene, a single walled carbon nanotube or a multi-walled carbon nanotube) a metal (e.g. iron, zinc, platinum, copper, sodium, cadmium, lanthanides, gadolinium, technetium, calcium, potassium, chromium, magnesium, molybdenum and salts or complexes thereof), a toxin (e.g. a ligand targeted toxin, a protease activated toxin, melittin and a toxin-based suicide gene therapeutic) or a nanoparticle (e.g. a metal nanoparticle such as gold, iron, silver, cobalt cadmium selenide, titanium oxide) or a core-shell metal nanoparticle such as CdS/ZnS, CdSe/ZnS, CdSe/CdS, and InAs/CdSe nanoparticle. Preferably, the nucleic acid is selected from the group comprising DNA, RNA, mRNA, siRNA, tRNA and micro-RNA. Preferably, the therapeutic agent is an enzyme associated with an over-expression in a metabolic disorder or disease or an under expression in a metabolic disorder or disease. Preferably, the enzyme is selected from the group comprising hydrogenase, dehydrogenase, lipase, lyase, ligase, protease, transferase, reductase, recombinase and nuclease acid modification enzyme. Preferably, the therapeutic agent is selected from the group comprising a cancer therapeutic, an anti-infection therapeutic, a vascular disease therapeutic, an immune therapeutic, senolytic and a neurological therapeutic. Preferably, the metal is selected from the group comprising iron, zinc, platinum, copper, sodium, cadmium, lanthanide, gadolinium, technetium, calcium, potassium, chromium, magnesium, molybdenum and salts or complexes thereof. Preferably, the toxin is selected from the group comprising a ligand targeted toxin, a protease activated toxin, melittin and a toxin-based suicide gene therapeutic.

Preferably, the guest cargo is a protein. Preferably a fluorescent protein. Preferably GFP, mCherry or mOrange. Preferably interleukin-2 (IL-2) or Neoleukin-2/15 (NL-2).

Preferably, wherein the number of TRAP rings in the TRAP-cage is between 6 and 60, preferably between 7 and 55, preferably between 8 and 50, preferably between 9 and 45, preferably between 10 and 40, preferably between 11 and 35, preferably between 12 and 34, preferably between 13 and 33, preferably between 14 and 32, preferably between 15 and 31, preferably between 16 and 30, preferably between 17 and 29, preferably between 18 and 28, preferably between 19 and 27, preferably between 20 and 26. Preferably the number of TRAP rings in the TRAP-cage is less than 40, preferably less than 35, preferably less than 30. Preferably the number of TRAP rings in the TRAP-cage is more than 6, preferably more than 10, preferably more than 15, preferably more than 20.

Preferably, the number of TRAP rings in the TRAP-cage is between 12 and 24.

Preferably, the number of TRAP rings in the TRAP-cage is about 24, preferably 24.

Preferably, the number of TRAP rings in the TRAP-cage is about 12, preferably 12.

Preferably, the number of TRAP rings in the TRAP-cage is about 20, preferably 20.

Preferably, the TRAP-cage according to the invention further includes an internal cargo encapsulated therein.

Preferably, opening of the cage is programmable. Preferably, said specific conditions corresponds to the specific cleavage characteristic of the cross-linker.

Preferably, the programmable opening of the cage is dependent on selection of a molecular or atomic metallic cross-linkers which hold the TRAP-rings in place in the TRAP-cage.

Preferably, the specific cleavage characteristic of the molecular cross-linker is selected from the group comprising:
(i) a reduction resistant/insensitive molecular cross-linker, whereby the cage remains closed under reducing conditions;
(ii) a reduction responsive/sensitive molecular cross-linker, whereby the cage opens under reducing conditions; and
(iii) a photoactivatable molecular cross-linker whereby the cage opens upon exposure to light.

Preferably, the reduction resistant/insensitive molecular cross-linker can be selected from the group comprising: bismaleimideohexane (BMH) and bis-bromoxylenes. Preferably, the reduction responsive/sensitive molecular cross-linker can be selected from the group comprising: dithiobismaleimideoethane (DTME). Preferably, the photoactivatable molecular cross-linker can be selected from the group comprising: bis-halomethyl benzene and its derivatives including 1,2-bis-bromomethyl-3-nitrobenzene (o-BBN), 2,4-bis-bromomethyl-1-nitrobenzene (m-BBN) and 1,3-bis-bromomethyl-4,6-dinitro-benzene (BDNB).

Preferably, the molecular cross-linker is a homobisfunctional molecular moiety and its derivatives. Preferably, homobisfunctional molecular cross-linker is bismaleimideohexane (BMH).

Preferably, the cage is resistant/insensitive to reducing conditions. Preferably the homobisfunctional molecular cross-linker is dithiobismaleimideoethane (DTME).

Preferably, the cage is responsive/sensitive to reducing conditions. Preferably the molecular cross-linker is a bis-halomethyl benzene and its derivatives.

Preferably, the molecular cross-linker is selected from the group comprising, 1, 2-bis-bromomethyl-3-nitrobenzene (BBN), bis-bromoxylene and 1,3-bis-bromomethyl-4,6-dinitro-benzene (BDNB).

Preferably, the molecular cross-linker is photolabile by exposure to UV light.

Preferably, the cage according to the invention comprises a mixture of different programmable molecular cross-linkers.

Preferably, the TRAP rings are variants.

Preferably, the artificial TRAP-cage protein is modified to comprise any one or more of the following mutations selected from the group comprising K35C, E48Q, E48K R64S, K35C/E48Q, K35C/E48K, and K35C/R64S. Preferably the artificial TRAP-cage protein is modified to comprise a K35C mutation. Preferably the artificial TRAP-cage protein is modified to comprise a K35C mutation or a K35C/E48Q mutation or a K35C/E48K mutation.

Preferably, the artificial TRAP-cage protein is modified to comprise any one or more of the following mutations selected from the group comprising K35C, K35H, R64S, K35C/R64S, K35H/R64S, S33C, S33H, S33C/R64S, S33H/R64S, S33C/K35H S33H/K35H, S33C/K35C, S33H/K35C.

Preferably, the TRAP-cages are stable in elevated temperatures, i.e. when the temperatures are elevated above normal room or human/animal body temperatures, preferably stable between 0 and 100° C., preferably stable between 15 and 100° C., preferably stable between 15 and 79° C., preferably stable up to 95° C., preferably stable at 95° C. and below.

Preferably, the TRAP-cages are stable in a non-neutral pH, preferably stable above pH 7 and below pH 7, preferably stable between pH 3 to 11, preferably stable between pH 4 to 10, preferably stable between pH 5 to 9.

Preferably, the TRAP-cages are stable in chaotropic agents (agents which disrupt hydrogen bonding in solution, which would disrupt or denature protein or macromolecular structures) or surfactants that would otherwise be expected to disrupt or denature protein or macromolecular structures. Preferably the cages show stability in n-butanol, ethanol, guanidinium chloride, lithium perchlorate, lithium acetate, magnesium chloride, phenol, 2-propanol, sodium dodecyl sulfate, thiourea, and urea. Preferably, the TRAP-cages are stable in up to 4 M GndHCl. Preferably, the TRAP-cages are stable in up to at least 7 M urea. Preferably, the TRAP-cages are stable in up to 15% of SDS. The stability of the cages described herein can be tested in standard conditions which would be known to the person of skill in the art using these agents to demonstrate said stability.

If no cysteine is present in the biomolecule, or they are present but not available for the reaction, —SH group, preferably as a group of cysteine, may be introduced into the biomolecule.

Introduction of cysteine can be carried out by any method known in the art. For example, but not limited to, the introduction of the cysteine is performed by methods known in the art, such as commercial gene synthesis or PCR-based site-directed mutagenesis using modified DNA primers. Above-mentioned methods are known by the persons skilled in the art and ready-to use kits with protocols are available commercially.

—SH moiety may be introduced into the biomolecule also by modification of other amino acids in the biomolecule i.e. by site-directed mutagenesis or by solid phase peptide synthesis.

The subject matter of the invention is also a TRAP-cage produced by this method. These cages may have any of the features or properties as described in relation to the first aspect of the invention, above, or anything else described herein.

The subject matter of the invention is also use of the cage according to the invention, as defined above, in delivery of a cargo in a controlled period and to a desired location.

The subject matter of the invention is also use of any of the TRAP-cages described herein as a medicament. The subject matter of the invention is also use of any of the TRAP-cages described herein as a vaccine.

The subject matter of the invention is also the use of any of the TRAP-cages described herein in treating a disease in a patient.

The subject matter of the invention is also a method of treating a patient, comprising administering the TRAP-cages described herein to said patient. The subject matter of the invention is also a method of treatment of an individual in need of therapy suffering from a condition selected from the group comprising cancer, vascular disease, cardiovascular disease, diabetes, infection, cellular senescence auto-immune condition, neurological/neurodegenerative disease, arthritis and respiratory disease, the method comprising administering a therapeutically effective amount of an artificial TRAP-cage bearing one or more external decorations selected from the group comprising nanobodies, antibodies, epitopes, antigens, proteins, peptides, cell penetrating peptides, antigenic peptides, polypeptides, nucleic acids, signaling molecules, lipids, oligosaccharides, dye molecules, inorganic nanoparticles, specific ligands and small molecule therapeutics or fragments thereof.

The subject matter of the invention is also a method of vaccinating an individual. Said individual may be suffering from a condition selected from the group comprising cancer, vascular disease, cardiovascular disease, diabetes, infection, cellular senescence, auto-immune conditions, neurological/neurodegenerative disease, arthritis and respiratory disease, the method comprising administering a therapeutically effective amount of an artificial TRAP-cage bearing one or more external decorations selected from the group comprising nanobodies, antibodies, epitopes, antigens, proteins, peptides, cell penetrating peptides, antigenic peptides, polypeptides, nucleic acids, signaling molecules, lipids, oligosaccharides, dye molecules, inorganic nanoparticles, specific ligands and small molecule therapeutics or fragments thereof.

Preferably the TRAP-cage therapeutic is administered via intranasal inhalation or injection.

### DETAILED DESCRIPTION OF THE INVENTION

Reference here to “TRAP protein” refers to Tryptophan RNA-binding attenuation protein, a bacterial protein. This protein can for example be isolated from wild type Geobacillus stearothermophilus, or other such bacteria. This protein can be isolated from various bacteria, but TRAP proteins which will work as described herein can be isolated from bacteria such as Alkalihalobacillus ligniniphilus, Anaerobacillus isosaccharinicus, Anoxybacillus caldiproteolyticus, Anoxybacillus calidus, Anoxybacillus pushchinoensis, Anoxybacillus tepidamans, Anoxybacillus tepidamans, Anoxybacillus vitaminiphilus, Bacillaceae bacterium, Bacillus alveayuensis, Bacillus alveayuensis, Bacillus sinesaloumensis, Bacillus sp. FJAT-14578, Bacillus sp. HD4P25, Bacillus sp. HMF5848, Bacillus sp. PS06, Bacillus sp. REN16, Bacillus sp. SA1-12, Bacillus sp. V3-13, Bacillus timonensis, Bacillus timonensis, Bacillus weihaiensis, Bacillus yapensis, Calidifontibacillus erzurumensis, Calidifontibacillus oryziterrae, Cytobacillus luteolus, Fredinandcohnia aciditolerans, Fredinandcohnia humi, Fredinandcohnia onubensis, Fredinandcohnia onubensis, Geobacillus genomo sp. 3, Geobacillus sp. 46C-IIa, Geobacillus stearomthermophilus, Geobacillus stearothermophilus, Geobacillus stearothermophilus, Geobacillus stearomthermophilus, Geobacillus stearothermophilus, Geobacillus stearomthermophilus, Geobacillus stearothermophilus, Geobacillus thermodenitrificans NG80-2, Halobacillus dabanensis, Halobacillus halophilus, Halobacillus halophilus, Jeotgalibacillus proteolyticus, Litchfieldia alkalitelluris, Litchfieldia salsa, Mesobacillus harenae, Metabacillus, Metabacillus litoralis, Metabacillus sediminilitoris, Oceanobacillus limi, Oceanobacillus sp. Castelsardo, Ornithinibacillus, Ornithinibacillus bavariensis, Ornithinibacillus contaminans, Ornithinibacillus halophilus, Ornithinibacillus scapharcae, Parageobacillus caldoxylosilyticus, Parageobacillus genomo sp., Parageobacillus thermantarcticus, Parageobacillus thermantarcticus, Parageobacillus thermoglucosidasius, Parageobacillus thermoglucosidasius, Paucisalibacillus globulus, Paucisalibacillus sp. EB02, Priestia abyssalis, Priestia endophytica, Priestia filamentosa, Priestia koreensis, Priestia megaterium, Psychrobacillus glaciei, Salinibacillus xinjiangensis, Sutcliftiella cohnii, Thermolongibacillus altinsuensis.

Trp RNA-binding attenuation protein is a bacterial, ring-shaped homo 11-mer (see A. A. Antson, J. Otridge, A. M. Brzozowski, E. J. Dodson, G. G. Dodson, K. S. Wilson, T. Smith, M. Yang, T. Kurecki, P. Gollnick, which is hereby incorporated by reference), The structure of trp RNA-binding attenuation, protein can be seen in the literature (Nature 374, 693-700 (1995), which is hereby incorporated by reference).

Suitably, the protein used herein is a modified version of wild-type TRAP isolated from Bacillus stearomthermophilus. This is seen in Table 1:

TABLE 1

| Name | Protein sequence |
|---|---|
| Wild-type TRAP | MYTNSDFVVIKALEDGVNVIGLTRGADTRFH |
| Bacillus | HSEKLDKGEVLIAQFTEHTSAIKVRGKAYIQ |
| stearothermophilus | TRHGVIESEGKK* |
| (PDB: 1QAW) | [SEQ ID NO: 1] |

The Wild-type TRAP Bacillus stearothermophilus gene sequence is seen in Table 2:

TABLE 2

| Name | Gene sequence | Gene ID (from UniProt) |
|---|---|---|
| Wild-type TRAP | atgtatacgaacagcgactttgttgtcattaa | 58572467 |
| Bacillus | agcgcttgaagacggagtgaacgtcattggat | |
| stearothermophilus | tgacgcgcggggcggatacacggttccatcac | |
| | tcggaaaagctcgataaaggcgaagtgttgat | |
| | cgcccagtttacagagcacacgtcggcgatta | |
| | aagtgagaggcaaggcgtatattcaaacgcgc | |
| | catggcgtcattgagtcggaagggaaaaagta | |
| | a | |
| | [SEQ ID NO: 2] | |

Preferably, preparation of proteins is performed by biomolecule expression in a suitable expression system and purification of the expression product. Preferably with a modified version of the above Wild-type TRAP Bacillus stearothermophilus gene sequence.

TRAP proteins forms rings, herein “TRAP rings”, and rings are the natural state of TRAP proteins. Typically, as is the case for the Geobacillus Stearothermophilus proteins as demonstrated herein, TRAP monomer proteins spontaneously assemble into toroids or rings made from monomers.

Reference herein to a “TRAP-cage lumen” is the hollow interior of the TRAP-cage. It is separated from the external environment by TRAP rings which form the wall of the TRAP-cage where any holes in this wall are considered to separate the lumen form exterior environment by a flat plane between the edges of the TRAP-rings lining the hole.

TRAP-cages only form under particular conditions, for example as demonstrated herein with the presence of cysteines that can be crosslinked resulting in rings assembling into a cage. For example, as demonstrated herein, these will form with the presence of cysteine at position 35 (the result of a K35C mutation).

Reference herein to “TRAP ring” is synonymous with a TRAP building block, a subunit of the TRAP-cage complex or a TRAP monomer assembly. Reference herein to an “analog” of a particular protein or nucleotide sequence refers to a protein or nucleotide sequence having sufficient identity or homology to the protein or nucleotide sequence to be able to carry out the specified function, e.g. TRAP-cage formation under the conditions described herein, or encode a protein which is able to carry out the specified function, e.g. TRAP-cage formation under the conditions described herein.

To determine the percent identity/homology of two sequences, the sequence in question and a reference are aligned for optimal comparison purposes (e.g., gaps can be introduced in one or both of a first and a second amino acid or nucleic acid sequence for optimal alignment and non-homologous sequences can be disregarded for comparison purposes). A sequence may be determined an analog of a particular when it has preferably at least 40%, more preferably at least 50%, even more preferably at least 60%, and even more preferably at least 70%, 75%, 80%, 82%, 84%, 85%, 86%, 87%, 88%, 89%, 90%, 91%, 92%, 93%, 94%, 95%, 96%, 97%, 98%, 99%, or 100% of the amino acids or nucleotides of the relevant lengths of the reference sequence. When the amino acid residues or nucleotides at corresponding amino acid positions or nucleotide positions are compared, when a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap.

Suitably, the TRAP protein comprises an amino acid sequence having at least 80%, preferably at least 85%, preferably at least 90%, preferably at least 95%, preferably at least 97% identity or homology to the amino acid sequence of SEQ ID NO: 1. Preferably, the TRAP protein comprises an amino acid sequence having at least at least 85% identity or homology to the amino acid sequence of SEQ ID NO: 1.

Reference herein to “TRAP-cage” refers to an assembled protein complex formed from multiple biomolecules, here multiple TRAP protein rings forming the complex. The TRAP protein rings can be linked together by crosslinkers, herein molecular cross-linkers. “Complex”, “assembly”, “aggregate”, are used alternatively in the description and means a superstructure constructed by the reaction between biomolecules. The amount of the units involved in the complex depends on the nature of the biomolecule. More specifically, it depends on the amount of the biomolecule and the amount of —SH groups present in the biomolecule. “TRAP-cage” and “artificial TRAP-cage” are used interchangeably herein.

TRAP protein is a suitable biomolecule model for the method of the invention. This is likely due to its high intrinsic stability, toroid shape, lack of native cysteine residues (for easier control of the conjugation process) and availability of a residue that can be changed to cysteines with the resulting cysteine being in a suitable chemical and spatial environment suitable for proper bond formation.

Reference herein to “programmable” is intended to convey that the TRAP-cages of the present invention have properties conferred on, or engineered into them that make them prone or susceptible or predisposed to behave in a particular and selected manner on exposure to specific environmental conditions or stimuli.

Reference herein to “open” is synonymous with the TRAP-cage, fracturing, leaking, fragmenting, breaking or generally allowing a cargo to escape from the interior of the cage.

Reference herein to “closed” is synonymous with the TRAP-cage remaining intact, unbreakable, impervious or generally remaining as a whole cage.

Reference herein to “bisfunctional” refers to a molecular crosslinker which has two functional groups, for example herein a molecule with two functional groups, where there is one functional group for each of the cysteine thiol groups to be crosslinked in order to connect TRAP rings in a TRAP-cage. Reference herein to “homobisfunctional” refers to a bisfunctional linker where the two groups are the same. Preferably, homobisfunctional linkers include bismaleimideohexane (BMH), dithiobismaleimideoethane (DTME), bis-halomethyl benzene and its derivatives, 2-bis-bromomethyl-3-nitrobenzene (BBN), bis-bromoxylene and 1,3-bis-bromomethyl-4,6-dinitro-benzene (BDNB).

“Molecular cross-linker” is a molecule that acts to connect units, subunits, molecules, biomolecules or monomers to other examples of the same via formation of one or more chemical bonds. Molecular crosslinkers are not single atoms linkers, which are distinct entities.

Reference herein to “decoration” refers to something attached to the outer surface or exterior of the TRAP-cage. This can be any of the entities or moieties described herein.

Reference herein to “exterior” refers to the outer surface of the TRAP-cage and the surface which, in vivo, is the surface presented to a host. Accordingly, any exterior decoration is thus presented to a host and can illicit an appropriate response.

Reference herein to “attached” refers to a physical or chemical bond of the exterior decoration to the exterior surface of the TRAP-cage. Reference herein to “covalently attached” refers to formation of a chemical bond between the TRAP cage and the attachment.

Reference herein to “chemical modification” refers to modification by a chemical reaction, i.e. formation of a covalent bond or covalent attachment of something to the TRAP-cage.

Reference herein to “enzymatic coupling” refers to attaching a decoration to the exterior of the cage via a covalent bond whose formation is catalysed by an enzyme.

Reference herein to “bio-conjugation” refers to attaching a non-biological molecule to a biological molecule e.g. a fluorescent dye.

Reference herein to “genetic coupling” refers to attachment by adding the gene sequence of the decorating peptide/protein to the gene sequence of TRAP such that the protein resulting from protein formation is a fusion protein, wherein the decorating molecule is located on the exterior surface of TRAP-cage once assembled. This can also be known as genetic fusion approach. “Genetic coupling” and “genetic fusion” are used interchangeably herein.

Attachment of molecules to the exterior of TRAP-cage can be carried out using the SpyTag/SpyCatcher technique (Zakeri, B. et al., “Peptide tag forming a rapid covalent bond to a protein, through engineering a bacterial adhesin” Proceedings of the National Academy of Sciences, 109, (2012): E690-E697, which is hereby incorporated by reference). In this approach a modified CnaB2 domain from Streptococcus pyogenes is used. This is split into a C-terminal beta strand (13 amino acids) known as SpyTag and the remainder of the protein, known as SpyCatcher. When mixed in solution, the two form an isopeptide bond. In this way a molecule or particle bearing the SpyTag can be mixed with a molecule or particle bearing SpyCatcher in solution and the two will become covalently attached via formation of the isopeptide bond. Typically for binding together of peptides/proteins, this is achieved by genetic fusion of the SpyTag sequence to the C-terminus of one partner peptide/protein and the addition of the DNA sequence encoding SpyCatcher to any location in the DNA sequence encoding other peptide/protein which will result in production of a correctly folded recombinant peptide/protein wherein the SpyCatcher is accessible for reaction with SpyTag. This can be adapted to provide multiple copies of a SpyCatcher protein facing the exterior surface of a TRAP-cage.

Reference herein to “click chemistry” refers to a method for attaching a probe or substrate of interest to a specific biomolecule, here a TRAP-cage. This is a form of bioconjugation. It usually consists of small molecule reactions allowing the joining of substrates of choice with the TRAP-cages. For example, Copper(I)-catalyzed azide-alkyne cycloaddition (CuAAC), Strain-promoted azide-alkyne cycloaddition (SPAAC) or Strain-promoted alkyne-nitrone cycloaddition (SPANC).

The decorations could also be antibody binding domains (preferably, variants Z15, Z34 and Z34c, all derived from Protein A, adhirons, anti-RBD domain of SARS-CoV-2 Spike protein. Preferably, the nanobodies are fluorescent protein (GFP)-nanobodies (a single-chain V_{H}H antibody domain developed with specific binding activity against GFP) or nanobodies (Nbs), an isolated, binding portion of an antibody. Preferably, the antibodies are antibodies targeting cell receptors or antibodies targeting cancer regulatory proteins such as anti-mutant p53 antibodies. Preferably, the proteins are receptor binding molecules, lectins, or transferring, transferrin receptor binding proteins. They may be fluorescent proteins, preferably mCherry, tdTomato, dTomato. Preferably, the peptides are peptide hormones, cell membrane disrupting peptides, T-cell-stimulating peptides or another type of peptides. Preferably, the nucleic acids are DNA, designed DNA nanostructures including those designed using the DNA origami technique, DNAzymes, RNA, mRNA, miRNA, siRNA, tRNA single stranded RNA, double stranded RNA, RNAzymes. Preferably, the nucleic acid is selected from the group comprising DNA, RNA, mRNA, siRNA, tRNA and micro-RNA. Preferably, the signaling molecules are steroid hormones, neurotransmitters, eicosanoids. Preferably, the lipids are phospholipids such as Phosphatidylcholine Preferably, the oligosaccharides are sucrose, fructose, or monosaccharides particularly glucose. Preferably, the dye molecules are fluorescent dyes. Preferably, the antigenic peptides are CpG dinucleotide motifs. Preferably, the inorganic nanoparticles are metal nanoparticles such as titanium oxide nanoparticles, iron, zinc, platinum, copper, sodium, cadmium, lanthanides, gadolinium, technetium, calcium, potassium, chromium, magnesium, molybdenum and salts or complexes thereof, or a carbon-based structure (e.g. a fullerene or a buckminsterfullerene, a single walled carbon nanotube or a multi-walled carbon nanotube).

The decoration could be something that could act recognised as an antigen, e.g. SARS-CoV-2 spike protein full length, SARS-CoV-2 spike protein, receptor binding domain, SARS-CoV-2 spike protein, peptides thereof, SARS-CoV-2 spike protein full length, SARS-CoV-2 spike protein, receptor binding domain, SARS-CoV-2 spike protein, peptides thereof, AARS-CoV-2 non-spike structural proteins, SARS-CoV-2 non-spike structural proteins, peptides thereof, SARS-Cov-2 genome encoded proteins or parts thereof, Respiratory Syncytial Virus spike protein full length, Respiratory Syncytial Virus spike protein, receptor binding domain, Respiratory Syncytial Virus spike protein, peptides thereof, Respiratory Syncytial Virus spike protein full length, Respiratory Syncytial Virus spike protein, receptor binding domain, Respiratory Syncytial Virus spike protein, peptides thereof, Respiratory Syncytial Virus non-spike structural proteins, Respiratory Syncytial Virus non-spike structural proteins, peptides thereof, Respiratory Syncytial Virus genome encoded proteins or parts thereof, Lassa virus spike protein full length, Lassa virus spike protein, receptor binding domain, Lassa virus spike protein, peptides thereof, Lassa virus spike protein full length, Lassa virus spike protein, receptor binding domain, Lassa virus spike protein, peptides thereof, Lassa virus non-spike structural proteins, Lassa virus non-spike structural proteins, peptides thereof, Lassa virus genome encoded proteins or parts thereof, Epstien-Barr virus spike protein full length, Epstien-Barr virus spike protein, receptor binding domain, Epstien-Barr virus spike protein, peptides thereof, Epstien-Barr virus spike protein full length, Epstien-Barr virus spike protein, receptor binding domain, Epstien-Barr virus spike protein, peptides thereof, Epstien-Barr virus non-spike structural proteins, Epstien-Barr virus non-spike structural proteins, peptides thereof, Epstien-Barr virus genome encoded proteins or parts thereof, Dengue Fever virus structural proteins N, M or E, Dengue Fever virus structural proteins N, M or E, peptides thereof, Dengue Fever virus structural proteins N, M or E, portions thereof, cytomegalovirus proteins, portions thereof and derived peptides including capsid proteins, tegument proteins, polymerases and other proteins encoded by the viral genome, Influenza Virus HA protein full length, Influenza Virus HA protein, receptor binding domain, Influenza Virus HA protein, peptides thereof, Influenza Virus non-HA structural proteins, Influenza Virus non-HA structural proteins, peptides thereof, Influenza Virus genome encoded proteins or parts thereof.

The decorations could be an antibody e.g. Anti-p53 antibody, an anti-mutant p53 antibody, an Anti-JAK mAb e.g. Tofacitinib and baricitinib, an Interleukin inhibitor e.g. tocilizumab, secukinumab and ustekinumab, an Anti-CD20 mAbs e.g. Rituximab, ofatumumab and ocrelizumab, an Anti-TNF mAb e.g. Infliximab, adalimumab and golimumab, an Anti-IgE mAb e.g. Omalizumab, Haemopoietic growth factors such epoetin, Anti-PD1 and PDL-1 mAb such Keytruda, Anti-CTLA4 mAb e.g. ipilimumab, Anti-IL2 antibodies, Anti-1112 antibodies, Anti-1115 antibodies, Anti-TGFBeta antibodies, Anti-angiogenesis mAb e.g. Avastin, Antagonist mAb of the A2A and A2B receptors, Anti-Her2 mAb e.g. Trastuzumab, Antibody dependent conjugates, Anti-EGFR mAb, Anti-VEGFR mAb, Anti-CD52 mAb e.g. Alemtuzumab, anti-BAFF mAb e.g. Belimumab, Anti-CD19 mAs e.g. Blinatumomab, Anti-CD30 mAb e.g. Brentuximab vedotin Anti-CD38 mAb e.g. Daratumumab, Anti-VEGFR2 mAb e.g. Ramucirumab or an Anti-IL6 mAb e.g. Siltuximab.

The decorations could be a lipid, such as phospholipids e.g. phosphatidylcholine, Phosphatidic acid (phosphatidate) (PA), Phosphatidylethanolamine (cephalin) (PE), Phosphatidylserine (PS), Phosphatidylinositol (PI0, Phosphatidylinositol phosphate (PIP), Phosphatidylinositol bisphosphate (PIP2) and Phosphatidylinositol trisphosphate (PIP3), (Sphingomyelin) (SPH) Ceramide phosphorylethanolamine (Sphingomyelin) (Cer-PE).

The decorations could be a peptide, such as a peptide hormone, a cell membrane disrupting peptide, a T-cell-stimulating peptide, or another type of peptide. The peptide hormone may be adrenocorticotropic hormone (ACTH), amylin, angiotensin, atrial natriuretic peptide (ANP), calcitonin, cholecystokinin (CCK), gastrin, ghrelin, glucagon, growth hormone, follicle-stimulating hormone (FSH), insulin, leptin, luteinizing hormone (LH), melanocyte-stimulating hormone (MSH), oxytocin, parathyroid hormone (PTH), prolactin, renin, somatostatin, thyroid-stimulating hormone (TSH), thyrotropin-releasing hormone (TRH), vasopressin, also called arginine vasopressin (AVP) or anti-diuretic hormone (ADH) or vasoactive intestinal peptide (VIP). The cell membrane disrupting peptide may be melittin. The T-cell-stimulating peptide may be an antigen such as the portions of antigen proteins described above. Another type of peptide may be Microcin B-17 and derivatives, Albicidin and derivatives, Peptide inhibitors of Myeloid cell leukemia 1 (mcl-1), pepstatin and derivatives thereof.

The decorations could be small molecule, such as antibiotic molecules e.g. a macrolide antibiotic, nicotinamide adenine dinucleotide (NAD+), nicotinamide mononucleotide, a chloresterol absorption inhibitor e.g. ezetimibe, a Fibrate e.g. gemfibrozil, bezafibrate and cipofibrate, HMG-CoA Reductase Inhibitor, Ranolazine, Ivabradine, a Nitrate such as glyceryl trinitrate, an Endothelin antagonist such as Bosentan, Hydralazine, Minoxidil, a Calcium channel blocker e.g. amlodipine, nifedipine, verapamil, diltiazem, an Angiotensin antagonist e.g. losartan, valsartan, candesartan and irbesartan, an ACE inhibitor e.g. captopril, enalapril, lisinopril, Digoxin, an Adenosine receptor agonist, a class IV Antidysrhythmic e.g. Verapamil Class Ill Antidysrhythmic e.g. Amiodarone, Class II Antidysrhythmic e.g. bisoprolol, esmolol and propranolol, Class I Antidysrhythmic e.g. Flecainide and Disopyramide, Anti-histamine e.g. Promethazine, cyclizine and Cetirizine, Glucocorticoid e.g. Prednisolone, dexamethasone and hydrocortisone, an Antiproliferative Immunosuppressant, an Calcineurin Inhibitor e.g. ciclosporin, an Uricosuric Agent e.g. Allopurinol and flebuxostat, a DMARD, a COX-2 Inhibitor e.g. Celecoxib, etoricoxib and parecoxib, a NSAID, a DOPA Decarboxylase Inhibitor e.g. Carbidopa or benserazide, a Selective B3-Adrenoceptor agonist, an a1-receptor agonist, a B1 receptor agonist e.g. Dobutamine, an a1 receptor antagonist e.g. prazosin, doxazosin and tamsulosin, a B2 receptor agonist e.g. salbutamol and terbutaline, a Nicotinic Partial Agonist e.g. Varenicline, a Peripheral Anticholinesterases e.g. Neostigmine, a Neuromuscular blocker e.g. panucuronium, vecuronium and rocuronium, a Bladder control drug e.g. oxybutynin and tolterodine, an Anti-metabolite e.g. folate antagonists, pyrimidine analogues and purine analogues, an Alkylating agent, an anti-fungal drug e.g. Grisofluvin, caspofungin and terbinafine, an anti-fungal antibiotic e.g. Amphotericin and nystatin, an Artemisinin Derivative e.g. artesunate and artemisinin, a Folate inhibitor e.g. proguanil, Primaquine, a Blood schizonticide e.g. chloquine and quinine, a Neuraminidase inhibitor e.g. Oseltamivir and zanamivir, a DNA Polymerase Inhibitor e.g. Aciclovir and glanciclovir, a Protease inhibitor e.g. Darunavir and ritanovir, a Reverse transcriptase inhibitor e.g. nevirapine and efavirenz, an Antiepileptic drug e.g. Carbamezepine, gabapentin, and pregabalin, a Tricyclic antidepressant e.g. amitriptyline nortriptyline and desipramine, an Opioid, a AMPA receptor Blocker e.g. Topiramate, a Barbiturate, a Benzodiazepin e.g. Lorazepam, midazolam and diazepam, a sodium channel inhibitors e.g. Carbamezepine, oxcarbazepine and phenytoin, a drug for bipolar disease e.g. lithium, a dopamine reuptake inhibitor e.g. Bupropion, a Monoamine oxidase inhibitor e.g. phenelzine, isocarboxcazid and moclobemide, a Noradrenaline reuptake inhibitor e.g. reboxetine and maprotiline, a SNRI e.g. venlafaxine, duloxetidne and desvenlafaxine, a SSRI e.g. fluoxetine, paroxetine, citalopram, escitalopram and sertraline, a Tricyclic e.g. imipramine and clomipramine, an Anti-pysychotic e.g. amisulpride and supiride, a Partial serotonin agonist, a NMDA receptor antagonist e.g. memantine, a Cholinesterase inhibitor e.g. donepezil, rivastigmine and galantamine, a Monoxidase inhibitor e.g. selegiline and rasagiline, a COMT inhibitors such as entacapone and tolcapone, a Dopamine agonists e.g. pramipexole and rotigotine, a Phosphodiesterase Type V inhibitor e.g. sildenafil and tadalafil, a Uterine stimulant e.g. misoprostal, ergometrine and oxytocin, a GnRH analogue and inhibitors, an Alpha-glucosidase inhibitor, a SGLT-2 inhibitor e.g. canagliflozin and empagliflozin, a Dipeptidyl Petidase Inhibitor e.g. sitagliptin, saxagliptin and linagliptin, a Proton pump inhibitor e.g. Omeprazole, lansoprazole and pantoprazole, an Inhaled glucocorticoid e.g. neclometasone and budesonide, a Inhaled muscarinic antagonist e.g. tiotropium and glycopyrronium, a Leukotriene antagonist e.g. montelukast, a Beta2-receptor agonist e.g. almetrol and formoterol, an Anticoagulant e.g. dabigratran, heparin and apixaban, a STING antagonist, an Inflamasome inhibitor, a Targeted oncology drug, a Protein kinase inhibitor, a Cell cycle inhibitor, a PROTAC and other promoter of protein degradation, PARP inhibitor e.g. Niraparib, a ALK inhibitor e.g. Alectinib, a HDAC inhibitor e.g. Belinostat, a MEK inhibitor e.g. Cobimetinib, a BRAF inhibitor e.g. Dabrafenib, EGFR inhibitor e.g. Erlotinib, a mTOR inhibitor e.g. Everolimus, a HER2 inhibitor e.g. Lapatinib, a FLT3 kinase inhibitor e.g. Midostaurin, a JAK inhibitor e.g. Tofacitinib or a BCL2 inhibitor e.g. Venetoclax.

The decorations could be chemokines such as CCL19, CCL21, CXCL9, CXCL10, CXCL11.

The decorations could be ligands for cell surface receptors such as kisspeptins, angiotensin II, thrombin, gastrin releasing peptide, N-formylpeptides.

Reference herein to a “guest cargo” refers to the biologic or whatever is encapsulated within the TRAP-cage.

The guest cargo could be a protein, preferably selected from the group comprising an enzyme (e.g. protease, a nuclease, hydrogenase, dehydrogenase, lipase, lyase, ligase, transferase, reductase, recombinase, nuclease acid modification enzyme. or other type of enzyme) an antigen, an antibody. Or the cargo is another type of protein biological macromolecule (e.g. a sterol, steroid or a fatty acid). Or the cargo is a lipid, a peptide (e.g. a peptide hormone, a cell membrane disrupting peptide, a T-cell-stimulating peptide or another type of peptides) a nucleic acid (e.g. DNA, designed DNA nanostructures including those designed using the DNA origami technique, DNAzymes, RNA, mRNA, miRNA, siRNA, tRNA single stranded RNA, double stranded RNA, RNAzymes), a small molecular cargo such as a drug, a peptide nucleic acids (PNA), a carbon-based structure (e.g. a fullerene or a buckminsterfullerene, a single walled carbon nanotube or a multi-walled carbon nanotube) a metal (e.g. iron, zinc, platinum, copper, sodium, cadmium, lanthanides, gadolinium, technetium, calcium, potassium, chromium, magnesium, molybdenum and salts or complexes thereof), a toxin (e.g. a ligand targeted toxin, a protease activated toxin, melittin and a toxin-based suicide gene therapeutic) or a nanoparticle (e.g. a metal nanoparticle such as gold, iron, silver, cobalt cadmium selenide, titanium oxide) or a core-shell metal nanoparticle such as CdS/ZnS, CdSe/ZnS, CdSe/CdS, and InAs/CdSe nanoparticle.

The enzyme could be a protease is selected from the group comprising Bromelain, Botulinum toxin A, thrombin Factor VIIA, Protein C, TEV protease, serine proteases including the SB, SC, SE, SF, SH, SJ, SK, SO, SP, SR, SS, ST, PA, PB PC and PE superfamilies and the S48, S62, S68, S71, S72, S79, S81 families. Including, specifically Lon-A peptidase, Clp protease, lactoferin, nucleoporin 125, cysteine proteases including CA, CD, CE, CF, CL, CM, CN, CO, CP, PA, PB. PC, PD, and PE superfamilies and C7, C8, C21, C23, C27, C36, C42, C53 and C75 families including specifically papain, cathepsin K, calpain, separase, adenain, sortase A and Hedhehog protein, aspartic proteases including AA, AC, AD, AE and AF superfamilies including specific examples as follows, BACE1, BACE2, Cathespin D, CathespinE Chymosin, Napsin-Ad, Nepenthesin, Pepsin, Presenilin, plasmepsins, threonine proteases including PB and PE superfamilies including specifically orbithine acyltransferase, glutamic proteases including G1 and G2 superfamilies, metalloproteinases including metalloexpeptidases and metalloendopeptidases.

The enzyme could be nuclease is selected from the group comprising endonucleases e.g. deoxcyribonuclease I; human endonuclease V, CRISPR associated proteins (including Cas9, Cas12, Cas13) with or without associated nucleic acids including guide RNA; AP endonuclease; flap endonuclease

The protein could be another type of enzyme, for example SUMO Activating Enzyme E1, a DNA repair enzymes e.g. DNA ligase, a DNA methyltransferases e.g. the m6A, m4C and m5C classes, a ten-eleven translocation methylcytosine dioxygenase, early growth response protein 1 (EGR1), Oxoguanine glycosylase, a Caspase e.g. E3 ubiquitin ligases including including pVHL, CRBN, Mdm2, beta-TrCP1, DCAF15, DCAF16, RNF114, c-IAP1, or an E1 ligase, an E2 ligase, DNA glycosylase, or a toxin e.g. ricin toxin A chain, Diptheria toxin and fragemnts thereof, a pore-forming toxins e.g. exotoxin A, α-hemolysin, Gyr-I, Myeloid cell leukemia 1 (Mci-1), a DNA polymerase including DNA polymerase β, polymerase δ and polymerase ε or an Enzyme replacement therapy enzyme e.g, Agalsidase beta, Agalsidase alfa, Imiglucerase, Taliglucerase alfa, Velaglucerase alfa, Alglucerase, Sebelipase alpha, Laronidase, Idursulfase, Elosulfase alpha, Galsulfase, Alglucosidase alpha.

The cargo could be something that could act recognised as an antigen, e.g. SARS-CoV-2 spike protein full length, SARS-CoV-2 spike protein, receptor binding domain, SARS-CoV-2 spike protein, peptides thereof, SARS-CoV-2 spike protein full length, SARS-CoV-2 spike protein, receptor binding domain, SARS-CoV-2 spike protein, peptides thereof, AARS-CoV-2 non-spike structural proteins, SARS-CoV-2 non-spike structural proteins, peptides thereof, SARS-Cov-2 genome encoded proteins or parts thereof, Respiratory Syncytial Virus spike protein full length, Respiratory Syncytial Virus spike protein, receptor binding domain, Respiratory Syncytial Virus spike protein, peptides thereof, Respiratory Syncytial Virus spike protein full length, Respiratory Syncytial Virus spike protein, receptor binding domain, Respiratory Syncytial Virus spike protein, peptides thereof, Respiratory Syncytial Virus non-spike structural proteins, Respiratory Syncytial Virus non-spike structural proteins, peptides thereof, Respiratory Syncytial Virus genome encoded proteins or parts thereof, Lassa virus spike protein full length, Lassa virus spike protein, receptor binding domain, Lassa virus spike protein, peptides thereof, Lassa virus spike protein full length, Lassa virus spike protein, receptor binding domain, Lassa virus spike protein, peptides thereof, Lassa virus non-spike structural proteins, Lassa virus non-spike structural proteins, peptides thereof, Lassa virus genome encoded proteins or parts thereof, Epstien-Barr virus spike protein full length, Epstien-Barr virus spike protein, receptor binding domain, Epstien-Barr virus spike protein, peptides thereof, Epstien-Barr virus spike protein full length, Epstien-Barr virus spike protein, receptor binding domain, Epstien-Barr virus spike protein, peptides thereof, Epstien-Barr virus non-spike structural proteins, Epstien-Barr virus non-spike structural proteins, peptides thereof, Epstien-Barr virus genome encoded proteins or parts thereof, Dengue Fever virus structural proteins N, M or E, Dengue Fever virus structural proteins N, M or E, peptides thereof, Dengue Fever virus structural proteins N, M or E, portions thereof, cytomegalovirus proteins, portions thereof and derived peptides including capsid proteins, tegument proteins, polymerases and other proteins encoded by the viral genome, Influenza Virus HA protein full length, Influenza Virus HA protein, receptor binding domain, Influenza Virus HA protein, peptides thereof, Influenza Virus non-HA structural proteins, Influenza Virus non-HA structural proteins, peptides thereof, Influenza Virus genome encoded proteins or parts thereof.

The cargo could be an antibody e.g. Anti-p53 antibody, an anti-mutant p53 antibody, an Anti-JAK mAb e.g. Tofacitinib and baricitinib, an Interleukin inhibitor e.g. tocilizumab, secukinumab and ustekinumab, an Anti-CD20 mAbs e.g. Rituximab, ofatumumab and ocrelizumab, an Anti-TNF mAb e.g. Infliximab, adalimumab and golimumab, an Anti-IgE mAb e.g. Omalizumab, Haemopoietic growth factors such epoetin, Anti-PD1 and PDL-1 mAb such Keytruda, Anti-CTLA4 mAb e.g. ipilimumab, Anti-IL2 antibodies, Anti-1112 antibodies, Anti-II15 antibodies, Anti-TGFBeta antibodies, Anti-angiogenesis mAb e.g. Avastin, Antagonist mAb of the A2A and A2B receptors, Anti-Her2 mAb e.g. Trastuzumab, Antibody dependent conjugates, Anti-EGFR mAb, Anti-VEGFR mAb, Anti-CD52 mAb e.g. Alemtuzumab, anti-BAFF mAb e.g. Belimumab, Anti-CD19 mAs e.g. Blinatumomab, Anti-CD30 mAb e.g. Brentuximab vedotin Anti-CD38 mAb e.g. Daratumumab, Anti-VEGFR2 mAb e.g. Ramucirumab or an Anti-IL6 mAb e.g. Siltuximab.

The protein could be another type of protein, for example Target-of-Rapamycin (TOR), GATA transcription factor Gaf1 (Gaf one), A TALE (Transcription activator-like effectors) protein, a Zinc finger protein, a Tumor suppressor protein including those involved in control of gene expression e.g. p16, signal transducers e.g. (TGF)-β; checkpoint control protein e.g. BRCA1, proteins involved in cell adhesion e.g. CADM1, DNA repair proteins e.g. p53, a transcription factor e.g. Yamanaka factors (Oct3/4, Sox2, Klf4, c-Myc), cytochrome c, BCL proteins including Bcl-2 (B-cell lymphoma 2), transcriptional control proteins e.g. NF-κB, a Cytokine including chemokines, interferons, interleukins Including interleukin-2 and artificial versions thereof), lymphokines, and tumour necrosis factors, a Heat shock protein including heat shock beta-one protein, a Growth factor e.g. GDF11, ubiquitin, a DNA double-strand break repair protein e.g. DNA ligase IIIα, a PCSK9 inhibitor e.g. evolocumab and alirocumab, a Brain-derived neurotrophic factor (BDNF) or Inhibitors of IL-5 e.g. mepolizumab and reslizumab.

The cargo could be another type of biological macromolecule (e.g. a sterol, steroid or a fatty acid). The sterol may be cholesterol. The steroid may be progesterone. The fatty acid may be a saturated fatty acid e.g. Caprylic acid, capric acid, lauric acid, myristic acid, palmitic acid, stearic acid, arachidic acid, behenic acid, lignoceric acid, cerotic acid or an unsaturated fatty acid e.g. Myristoleic acid, Palmitoleic acid, Sapienic acid, Oleic acid, Elaidic acid, Vaccenic acid, Linoleic acid, Linoelaidic acid, α-Linolenic acid, Arachidonic acid, Eicosapentaenoic acid, Erucic acid, Docosahexaenoic acid.

The cargo could be a lipid, such as phospholipids e.g. phsophotdiylcholine, Phosphatidic acid (phosphatidate) (PA), Phosphatidylethanolamine (cephalin) (PE), Phosphatidylserine (PS), Phosphatidylinositol (PI0, Phosphatidylinositol phosphate (PIP), Phosphatidylinositol bisphosphate (PIP2) and Phosphatidylinositol trisphosphate (PIP3), (Sphingomyelin) (SPH) Ceramide phosphorylethanolamine (Sphingomyelin) (Cer-PE).

The cargo could be a peptide, such as a peptide hormone, a cell membrane disrupting peptide, a T-cell-stimulating peptide, or another type of peptide. The peptide hormone may be adrenocorticotropic hormone (ACTH), amylin, angiotensin, atrial natriuretic peptide (ANP), calcitonin, cholecystokinin (CCK), gastrin, ghrelin, glucagon, growth hormone, follicle-stimulating hormone (FSH), insulin, leptin, luteinizing hormone (LH), melanocyte-stimulating hormone (MSH), oxytocin, parathyroid hormone (PTH), prolactin, renin, somatostatin, thyroid-stimulating hormone (TSH), thyrotropin-releasing hormone (TRH), vasopressin, also called arginine vasopressin (AVP) or anti-diuretic hormone (ADH) or vasoactive intestinal peptide (VIP). The cell membrane disrupting peptide may be melittin. The T-cell-stimulating peptide may be an antigen such as the portions of antigen proteins described above. Another type of peptide may be Microcin B-17 and derivatives, Albicidin and derivatives, Peptide inhibitors of Myeloid cell leukemia 1 (mci-1), pepstatin and derivatives thereof.

The cargo could be a small molecular cargo, such as antibiotic molecules e.g. a macrolide antibiotic, nicotinamide adenine dinucleotide (NAD+), nicotinamide mononucleotide, a cholersterol absorption inhibitor e.g. ezetimibe, a Fibrate e.g. gemfibrozil, bezafibrate and cipofibrate, HMG-CoA Reductase Inhibitor, Ranolazine, Ivabradine, a Nitrate such as glyceryl trinitrate, an Endothelin antagonist such as Bosentan, Hydralazine, Minoxidil, a Calcium channel blocker e.g. amlodipine, nifedipine, verapamil, diltiazem, an Angiotensin antagonist e.g. losartan, valsartan, candesartan and irbesartan, an ACE inhibitor e.g. captopril, enalapril, lisinopril, Digoxin, an Adenosine receptor agonist, a class IV Antidysrhythmic e.g. Verapamil Class Ill Antidysrhythmic e.g. Amiodarone, Class II Antidysrtythmic e.g. bisoprolol, esmolol and propranolol, Class I Antidysrhythmic e.g. Flecainide and Disopyramide, Anti-histamine e.g. Promethazine, cyclizine and Cetirizine, Glucocorticoid e.g. Prednisolone, dexamethasone and hydrocortisone, an Antiproliferative Immunosuppressant, an Calcineurin Inhibitor e.g. ciclosporin, an Uricosuric Agent e.g. Allopurinol and flebuxostat, a DMARD, a COX-2 Inhibitor e.g. Celecoxib, etoricoxib and parecoxib, a NSAID, a DOPA Decarboxylase Inhibitor e.g. Carbidopa or benserazide, a Selective B3-Adrenoceptor agonist, an a1-receptor agonist, a B1 receptor agonist e.g. Dobutamine, an a1 receptor antagonist e.g. prazosin, doxazosin and tamsulosin, a B2 receptor agonist e.g. salbutamol and terbutaline, a Nicotinic Partial Agonist e.g. Varenicline, a Peripheral Anticholinesterases e.g. Neostigmine, a Neuromuscular blocker e.g. panucuronium, vecuronium and rocuronium, a Bladder control drug e.g. oxybutynin and tolterodine, an Anti-metabolite e.g. folate antagonists, pyrimidine analogues and purine analogues, an Alkylating agent, an anti-fungal drug e.g. Grisofluvin, caspofungin and terbinafine, an anti-fungal antibiotic e.g. Amphotericin and nystatin, an Artemisinin Derivative e.g. artesunate and artemisinin, a Folate inhibitor e.g. proguanil, Primaquine, a Blood schizonticide e.g. chloquine and quinine, a Neuraminidase inhibitor e.g. Oseltamivir and zanamivir, a DNA Polymerase Inhibitor e.g. Aciclovir and glanciclovir, a Protease inhibitor e.g. Darunavir and ritanovir, a Reverse transcriptase inhibitor e.g. nevirapine and efavirenz, an Antiepileptic drug e.g. Carbamezepine, gabapentin, and pregabalin, a Tricyclic antidepressant e.g. amitriptyline nortriptyline and desipramine, an Opioid, a AMPA receptor Blocker e.g. Topiramate, a Barbiturate, a Benzodiazepin e.g. Lorazepam, midazolam and diazepam, a sodium channel inhibitors e.g. Carbamezepine, oxcarbazepine and phenytoin, a drug for bipolar disease e.g. lithium, a dopamine reuptake inhibitor e.g. Bupropion, a Monoamine oxidase inhibitor e.g. phenelzine, isocarboxcazid and moclobemide, a Noradrenaline reuptake inhibitor e.g. reboxetine and maprotiline, a SNRI e.g. venlafaxine, duloxetidne and desvenlafaxine, a SSRI e.g. fluoxetine, paroxetine, citalopram, escitalopram and sertraline, a Tricyclic e.g. imipramine and clomipramine, an Anti-pysychotic e.g. amisulpride and supiride, a Partial serotonin agonist, a NMDA receptor antagonist e.g. memantine, a Cholinesterase inhibitor e.g. donepezil, rivastigmine and galantamine, a Monoxidase inhibitor e.g. selegiline and rasagiline, a COMT inhibitors such as entacapone and tolcapone, a Dopamine agonists e.g. pramipexole and rotigotine, a Phosphodiesterase Type V inhibitor e.g. sildenafil and tadalafil, a Uterine stimulant e.g. misoprostal, ergometrine and oxytocin, a GnRH analogue and inhibitors, an Alpha-glucosidase inhibitor, a SGLT-2 inhibitor e.g. canagliflozin and empagliflozin, a Dipeptidyl Petidase Inhibitor e.g. sitagliptin, saxagliptin and linagliptin, a Proton pump inhibitor e.g. Omeprazole, lansoprazole and pantoprazole, an Inhaled glucocorticoid e.g. neclometasone and budesonide, a Inhaled muscarinic antagonist e.g. tiotropium and glycopyrronium, a Leukotriene antagonist e.g. montelukast, a Beta2-receptor agonist e.g. almetrol and formoterol, an Anticoagulant e.g. dabigratran, heparin and apixaban, a STING antagonist, an Inflamasome inhibitor, a Targeted oncology drug, a Protein kinase inhibitor, a Cell cycle inhibitor, a PROTAC and other promoter of protein degradation, PARP inhibitor e.g. Niraparib, a ALK inhibitor e.g. Alectinib, a HDAC inhibitor e.g. Belinostat, a MEK inhibitor e.g. Cobimetinib, a BRAF inhibitor e.g. Dabrafenib, EGFR inhibitor e.g. Erlotinib, a mTOR inhibitor e.g. Everolimus, a HER2 inhibitor e.g. Lapatinib, a FLT3 kinase inhibitor e.g. Midostaurin, a JAK inhibitor e.g. Tofacitinib or a BCL2 inhibitor e.g. Venetoclax.

“Unit” “subunit” “molecule”, “biomolecule”, “monomer” are used alternatively in the description and means one molecule which connects to another molecule for the complex formation.

“Complex”, “assembly”, “aggregate”, are used alternatively in the description and means a superstructure constructed by the reaction between biomolecules. The amount of the units involved in the complex depends on the nature of the biomolecule. More specifically, it depends on the amount of the biomolecule and the amount of —SH groups present in the biomolecule.

Reference herein to a “Reduction resistant/insensitive molecular cross-linker” is reference to a cross-linker which is not cleaved by reduction reaction such as that typically seen when a disulphide bond is cleaved by a reducing agent. These cross-linkers are stable under conditions that would result in breaking of reduction sensitive bonds. These bismaleimideohexane (BMH) and bis-bromoxylenes.

Reference herein to a “Reduction responsive/sensitive molecular cross-linker” is reference to a cross-linker which is cleaved by reduction reaction such as that typically seen when a disulphide bond is cleaved by a reducing agent. These cross-linkers are not stable under conditions that would result in breaking of reduction sensitive bonds. These include dithiobismaleimideoethane (DTME).

Reference herein to a “Photoactivatable molecular cross-linker” is reference to a cross-linker that is photoreactive or sensitive to light, i.e. one that will be cleaved when exposed to light. This light can be UV or other such light of a specific range of wavelengths. These include, 2-bis-bromomethyl-3-nitrobenzene (o-BBN), 2,4-bis-bromomethyl-1-nitrobenzene (m-BBN) and 1,3-bis-bromomethyl-4,6-dinitro-benzene (BDNB).

Moreover, following abbreviations have been used: TRAP (trp RNA-binding attenuation protein), GFP (green fluorescence protein), PTD4 (protein transduction domain), CPP (cell penetrating peptide), SDS-PAGE (sodium dodecyl sulfate-polyacrylamide gel electrophoresis), TEM (transmission electron microscopy), DMEM (Dulbecco's Modified Eagle Medium), FBS (foetal bovine serum).

TRAP cages are amenable to chemical modification. The Au(I)-mediated TRAP-cage assembly possesses 24 free cysteines per cage, four at each of the six at the 4-fold symmetrical pore regions. These cysteines have been used for labelling the cages with Alexa-647 fluorescent dye containing a maleimide moiety (FIG. 1a). The chemical modification was performed using the TRAP-cages that were loaded with a negatively supercharged variant of green fluorescent protein, GFP(−21) (FIG. 1a). The amount of Alexa-647-maleimide (which was equal to the number of TRAP cysteine groups) to be added has been optimized, where the TRAP-cage is readily labelled and no free dye is present in the sample, noting concentrations that were unsuccessful (FIG. 1b).

TRAP naturally has three surfaces exposed lysines per monomer, corresponding to 792 lysines on the assembled cage, that are ready to react with many electrophile groups such as activated esters to form covalent bonds. To exploit this, the C-terminus of a model peptide PTD4 (YARAAARQARA), an optimised HIV TAT-based cell-penetrating peptide, has been converted to N-hydroxysuccinimide (NHS). This PTD4 derivative, Ac-YARAAARQARAG, has been attached to the amino groups on the surface-exposed lysines of TRAP-cages (FIG. 1a). The C-terminus of the peptide was activatedwith a sulfonated NHS using 1-ethyl-3-(3-dimethylaminopropyl) carbodiimide (EDC).

The assembled and purified TRAP cage was simply mixed with the peptide-NHS in 50 mM HEPES, 150 mM NaCl, pH 7.5 at room temperature for 2.5 hours. Native-PAGE analysis of the resulting mixture showed a substantial mobility shift compared to unmodified TRAP cage, suggesting successful cage modification with the peptide (FIG. 1c). Negative stain TEM confirmed that modified cages remain intact (FIG. 1d).

Efficient lysine modification in aqueous solution can be achieved with the compound containing an isothiocyanate moiety to yield a thiourea bond. This possibility has been demonstrated through TRAP-cage modification with fluorescein isothiocyanate (FITC) (FIG. 2). Furthermore, TRAP modification with peptides can be achieved not only with NHS esters to form amide bonds with lysines, but also via cysteine-modification using maleimide-based conjugation. As such, a PTD4 peptide derivative having a maleimide moiety at the N-terminus has been prepared. Successful decoration with this maleimidyl peptide was confirmed by native-PAGE analysis using TRAP cages encapsulating guest mCherry proteins in the lumen via genetic fusion strategy (FIG. 3).

Moreover, TRAP cages are amenable to enzymatic modification with peptide/protein. Despite the easiness of the modification using activated ester, this method is limited to peptides which do not contain any nucleophile amine and carboxylate in the sequence. In order to overcome this issue, we next employed an enzymatic coupling system using a peptide ligase (FIG. 4). Sortase A (SrtA) is a bacterial transpeptidase that catalyzes the reaction of fusing the LPXTG protein motif to a N-terminal polyglycine chain, yielding a fusion LPXT(G)ₙ¹. We equipped the TRAP^{K35C} C-terminus, which exposed to the exterior when assembled to cages, with the polypeptide containing LPSTG, yielding TRAP^{K35C}-srt (FIG. 4b). Like the parent protein, thismodified variant can assemble into cage-like structures upon addition of Au(I), judged by negative-stain transmission electron microscopy (TEM) (FIG. 4c). As initial models to decorate the TRAP cage, we selected four fluorescent proteins, mCherry, tdTomato, dTomato and dsRed2. These proteins, although their monomer units have similar protein folding, have different sizes and quaternary states (FIG. 4d), allowing us to check the influence of these factors on the reaction with TRAP cages. These model proteins were appended with a hexahistidine, a recognition sequence of the protease from tobacco etch virus (TEV protease), and a pentaglycine at the N-terminus (FIG. 4e), and recombinantly produced using E. coli cells. After purification and cleavage of the tag using TEV protease, the resulting proteins possessing an N-terminal pentaglycine was mixed with cages composed of TRAP-srt in the presence of SrtA. Analysis of the reaction mixtures using native-PAGE showed a substantial band shift compared to that of unmodified TRAP cages, suggesting successful modification of the protein cages with all the guest proteins (FIG. 3a). After isolation of the decorated TRAP cages using size-exclusion chromatography, the samples were further analyzed by dynamic light scattering (DLS), showing an increase in the diameter of the modified cage in proportion to the size of the decoration protein; 24.38 nm, 29.71 nm, 32.38 nm, 29.55 nm, and 50.96 nm for TRAP^{K35C}-srt cages, ones modified with mCherry, tdTomato, dTomato, and DsRed2, respectively. The exterior decoration and intact cage structure upon enzymatic was also confirmed by TEM (FIG. 3b). The differences between the model proteins turned out not to have a significant impact on the modification process, suggesting the possibility of using this method to obtain a wide spectrum of modifications with various proteins.

To further demonstrate the utility of the srtA-mediated decoration of TRAP cages, we chose nanobodies (Nbs), an isolated, binding portion of an antibody originally sourced from camelid single domain antibodies as next models (Muyldermans S. Nanobodies: natural single-domain antibodies. Annu Rev Biochem. 2013; 82:775-797. doi:10.1146/annurev-biochem-063011-092449, which is hereby incorporated by reference). Nbs are currently of great interest due to their high stability, easy expression in bacterial systems, small size and excellent binding affinity. However, their small size leads to quick filtration in the kidney, a marked disadvantage in the potential medical usage. We hypothesized that modification on the protein cage exterior can extend the lifetime of Nbs in blood stream. Additionally, multiple nanobodies displayed on a single particle may increase the avidity of binding to target. Nanobodies displayed on the exterior of protein cages could conceivably be used to localise cages and their therapeutic cargoes specifically at sites of interest e.g. receptors overexpressed on cancer cells. A GFP-binding Nb was used to facilitate the functional evaluation upon modification on the TRAP cage exterior. SDS- and native-PAGE analysis of the reaction with TRAP-srt cages in the presence of SrtA suggested successful exterior decoration with Nbs via covalent bond formation (FIG. 6a). Samples were further analyzed by DLS, showing an increase in the diameter of the modified cage; 26.21 nm; 30.65 nm, ones modified with Nbs and Nbs with further addition of GFP, respectively. Furthermore, upon the modification, the anti-GFP Nbs still retained the ability to bond with GFP (FIG. 6b).

Summarizing, TRAP cages are amenable to both chemical and enzymatic modification with peptide/protein. Likewise, many other molecules/materials such as DNAs, lipids, oligosaccharides, synthetic polymers and metal nanoparticles could be attached on TRAP cage exterior by introducing either NHS ester or polyglycine units in the structure for ester bond or sortase-mediated attachment respectively. Such robust and general exterior decoration strategies will contribute largely to drug carrier and vaccine development based on artificial protein cages.

According to an aspect, the cages as described herein may be used as medicaments. This could be in a of treating a patient, such as comprising administering a cage as described herein to a patient, or the cages as described herein for use in treating a disease in a patient. This particularly may be a cage designed to carry cargo or an external decoration and deliver, or possibly disassemble in presence of reducing agents for intracellular delivery. These cages may be administered along with or in the presence of a pharmaceutically acceptable carrier, adjuvant or excipient. The cargo that the cages for use as a medicament or for treating patients will be of benefit to said patient. For example, as drug delivery systems (DDS)—for active molecules (especially biological macromolecules such as RNA, DNA, peptides and proteins). They provide advantages as biological macromolecules are often easily disrupted or digested by conditions such as those found in vivo. Biological macromolecules are too big to diffuse out of the holes in TRAP, being a large protein, TRAP-cage can sustain significant changes without disrupting overall structure. This means that it can be modified to capture therapeutic cargoes and simultaneously be modified, on the exterior to target therapeutic targets. Programmable linkers can be used which cleave in a desired situation that correlates with arrival at site of action. For example, light could be shone on the target site to cleave open photocleavable TRAP-cages. If TRAP-cages penetrate cells, those held together by reducible linkers will spontaneously open up and release cargo as the cytoplasm of the cell is highly reducing. Cages could also be used in conjunction with vaccines or acting as vaccines, where antigens (i.e. peptides) which are expected to stimulate a T-cell response are captured inside the TRAP-cage and then targeted at to T-cells, followed by triggered opening.

### BRIEF DESCRIPTION OF THE FIGURES

FIG. 1. TRAP cage decoration via cysteine and lysine modification. (a) Scheme of TRAP-cage encapsulation with GFP(−21) and external modifications with Alexa-647 dye and PTD4 peptide. (b) Native PAGE gels showing TRAP-cage carrying GFP(−21) after titration of Alexa-647 in the conjugation reaction. Gels were analysed by fluorescence detection of Alexa-647 (left panel, exct. 647) and stained for proteins (right panel). Arrows show optimal decoration conditions used in further experiments. (c) Native PAGE gels showing encapsulation of GFP(−21) by unmodified TRAP-cage or TRAP-cage externally modified by Alexa-647 and PTD4. Lane 1: TRAP-cage with GFP(−21); 2: TRAP-cage with GFP(−21) decorated with Alexa-647; 3: TRAP-cage with GFP(−21) decorated with Alexa-647 and PTD4; 4: molecular weight marker for native PAGE. Gels were stained for protein (upper panel) and analysed by fluorescence detection of GFP (middle panel, exct. 488 nm) and Alexa-647 (bottom panel, exct. 647). (d) Negative stain transmission electron microscopy of TRAP-cage with GFP(−21) (left panel); TRAP-cage with GFP(−21) decorated with Alexa-647(middle panel); TRAP-cage with GFP(−21) decorated with Alexa-647 and PTD4 (right panel).

FIG. 2. External decoration of TRAP-cage with FITC dye. (a) Schematic for reaction of TRAP-cage with FITC. (b) Native PAGE gels showing TRAP-cage after conjugation with FITC dye. Gels were stained for proteins (left panel) and analysed by fluorescence detection of FITC (right panel, exct. 488). Lanes: 1: molecular weight marker for Native PAGE electrophoresis; 2: TRAP-cage with FITC in PBS buffer; 3: TRAP-cage with FITC in carbonate-biscarbonate buffer; 4: TRAP-cage with FITC in DMEM.

FIG. 3. External decoration of TRAP-cage filled with mCherry with 6-maleimide hexanoic-PTD4 peptides. (a) Schematic representation of cysteine-mediated TRAP-cage decoration. (b) Native PAGE gels showing TRAP-cage carrying mCherry after titration of PTD4 in the conjugation reaction. TRAP-cage after conjugation with PTD4 peptides.

FIG. 4. Concept and strategy of sortase-mediated TRAP cage decoration. (a) Schematic representation of sortase-mediated TRAP cage decoration with guest molecules. (b) The construct of TRAPK^{35C} possessing C-terminal sortase recognition sequence, TRAP^{K35C}-srt. (c) TEM images of Au(I)-induced assemblies composed of TRAP^{K35C}-srt and srtA-mediated decoration at the exterior. The samples were stained with 2% uranyl acetate. (d) Quaternary states of four red fluorescent proteins used in this study. (e) The construct design of model red fluorescent protein possessing a pentaglycine at the N-terminus.

FIG. 5. Sortase-mediated TRAP cage decoration with model fluorescent proteins. Native-PAGE analysis (a) and (b) TEM imaging of TRAPK^{35C}-srt cages modified with mCherry (mCh), tdTomato (tdT), dTomato (dT) dsRed2 (dsR2), Nanobodies (Nbs) and Nanobodies with further addition of GFP (Nbs-GFP). The protein bands on the gel was visualized using Instant Blue staining. For TEM, the samples were stained with 2% uranyl acetate.

FIG. 6. Sortase-mediated TRAP cage decoration with nanobodies. (a,b) SDS-PAGE (a) and nativePAGE (b) analysis of sortase mediated conjugation between TRAP^{K35C}-srt (9.5 kDa) cage and anti-GFP Nbs possessing an N-terminal pentaglycine (G5-Nbs^{GFP}, 13.2 kDa). Theoretical molecular mass of conjugated product is 22.65 kDa. The ability of the resulting TRAP cage displaying Nbs to bind with GFP was also confirmed (b, rightmost lane). These gels were visualized by Instant Blue staining.

### EXAMPLES

### Example 1. Chemical Modification of TRAP Cages

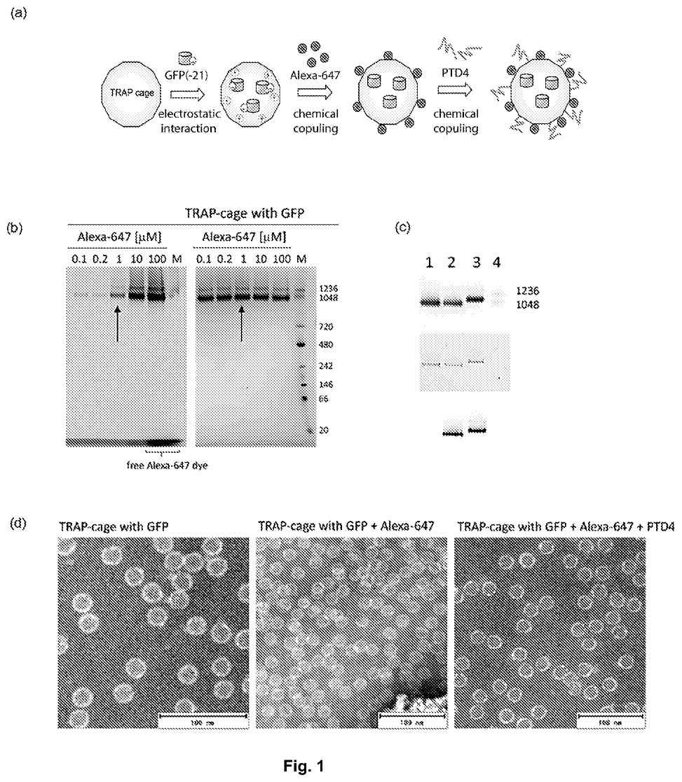

TRAP-cage carrying GFP labeling with Alexa-647 and decorated with cell-penetrating peptide Alexa Fluor-647 C2 maleimide fluorescent dye (Alexa-647, Thermo Fisher Scientific) and cell-penetrating PTD4 peptide were conjugated to the TRAP-cage filled with GFP via a crosslinking reactions with cysteines and lysines present in the TRAP protein (FIG. 1a).

To achieve fluorescent labelling, TRAP-cage carrying GFP was mixed with a Alexa-647 C2 maleimide dye, the reaction was conducted in 50 mM HEPES with 150 mM NaCl pH 7.5 for 2.5 h at room temperature with continuous stirring at 450 rpm. The optimal interaction ratio of maleimide-conjugated Alexa-647 to TRAP-cage was assessed by titration (FIG. 1b). Briefly, aliquots of TRAP-cage loaded with GFP(−21) were mixed with maleimide-conjugated Alexa-647 ranging from 0.1 μM to 100 μM. Samples were then separated by native gel electrophoresis and visualized by fluorescence detection in a Chemidoc, with excitation at 647 nm. Reactions where nofree Alexa-647 is present in the sample, were considered as optimal decoration conditions.

For the cell-penetrating peptide decoration, the peptide chain was constructed on resin using standard Fmoc-based solid phase peptide synthesis (SPPS) using a N,N′-diisopropylcarbodiimide (DIC)/Oxyma coupling system and the N-terminus was capped using acetic anhydride. After cleavage from the resin and deprotection, the peptide was purified by reverse-phase high performance liquid chromatography (RP-HPLC). Purified PTD4 peptide was mixed with 1-ethyl-3-(-3-dimethylaminopropyl) carbodiimide hydrochloride (EDC, 10 μl, 83 mM) and N-hydroxysuccinimide (NHS, 10 μl, 435 mM), all reagents dissolved in ddH₂O. Subsequently, the excess of activated PTD4 peptides were added to TRAP-cage filled with GFP(−21) and labelled with Alexa-647 and incubated for next 2.5 h at room temperature, with continuous stirring at 450 rpm. The reaction was stopped by addition of 5 μl of 200 mM Tris-HCl pH 7.5. The conjugation efficiency was verified by native PAGE and fluorescent gel imaging. A change in molar weight of the decorated TRAP-cage results in a band shift observed in native PAGE (FIG. 1c). Negative stain transmission electron microscopy (TEM) confirmed that the modified TRAP-cages retained their characteristic shape (FIG. 1d).

TRAP-Cage Labeling with FITC (Fluorescein Isothiocyanate) Dye

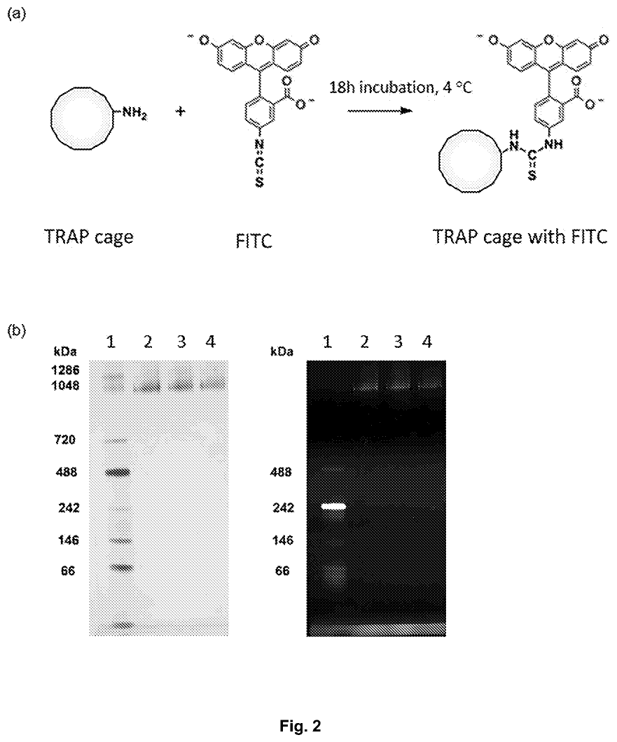

FITC (fluorescein isothiocyanate) fluorescent dye (FITC, Sigma) was conjugated to the TRAP-cage via reactions with lysines present in the TRAP protein. To achieve fluorescent labelling, TRAP-cage (200 μl, 0.5 mg/ml nM) was mixed with a FITC dye (50 μl, 0.25 mg/ml), the reaction was conducted in 0.1 M sodium carbonate-bicarbonate buffer, pH 9.0, for overnight, 4° C. with gentle stirring. Excess of FITC dye was removed using the Sephadex G-25M column following the manufacturers recommended protocol. Samples were subsequently analyzed by native PAGE followed by Instant blue gel staining and visualized by fluorescence detection in a Chemidoc, with excitation at 488 nm (FIG. 2b).

TRAP-Cage with mCherry Decoration with Cell-Penetrating Peptides

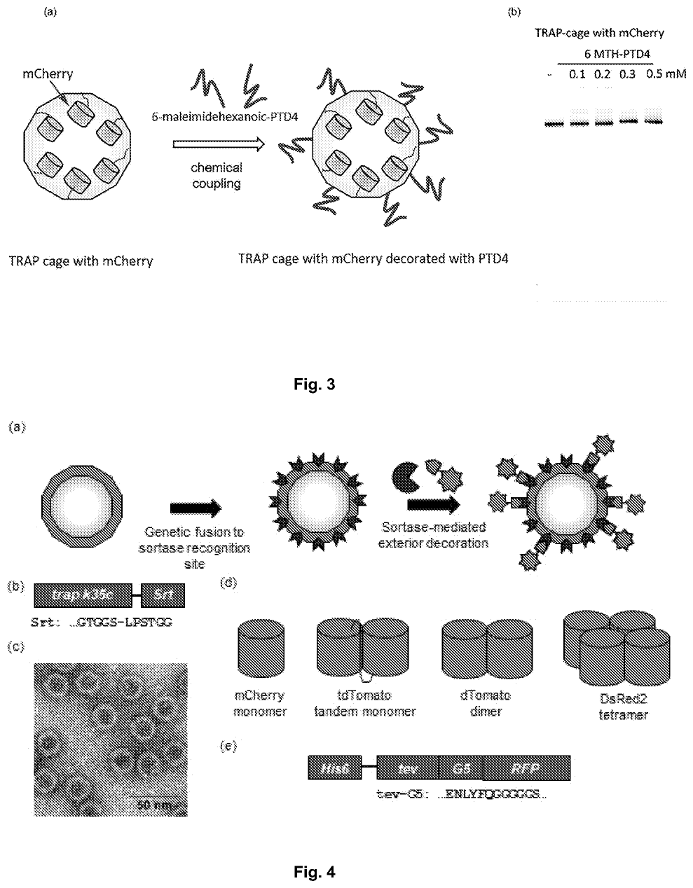

A maleimide moiety was introduced at the N-terminus of the peptide on resin using 6-maleimide hexanoic acid and a DIC/Oxyma coupling protocol. The 6-maleimide hexanoic-PTD4 peptide ranging from 0.1 μM to 0.5 mM was mixed with TRAP-cage filled with mCherry (100 μl, 0.3 mg/ml) and incubated overnight at room temperature, with continuous stirring at 450 rpm. The conjugation efficiency was verified by native PAGE and fluorescent gel imaging. A change in molar weight of the decorated TRAP-cage results in a band shift observed in native PAGE (FIG. 3b).

### Example 2. Enzymatic Modification of TRAP Cages

### Protein Design, Production and Purification

The TRAP cages were obtained as described previously (Malay, Ali D., et al. “An ultra-stable gold-coordinated protein cage displaying reversible assembly.” Nature 569.7756 (2019): 438-442), with the TRAP variant having K35C mutation and the appended amino acid sequence of GTGGSLPSTG at the C-terminus. SrtA gene wasordered from commercial vendor (BioCat), already subcloned into pET30b(+) plasmid.

E. coli strain BL21 (DE3) cells were transformed with the plasmid and precultured in LB medium at 37° C. until the OD600 value reached to ˜0.6 at which point protein expression was induced by addition of isopropyl β-d-1-thiogalactopyranoside (IPTG) to a final concentration of 0.5 mM, followed by further cell culture at 25° C. overnight. After cell lysis by sonication, these proteins were purified by Ni-NTA affinity chromatography and size-exclusion chromatography using a Superdex 200 Increase 10/300 column (GE Healthcare). The genes of the fluorescent proteins (mCherry, tdTomato, dTomato, dsRed2) and nanobodies (anti-GFP nanobodies) were modified with genes encoding a 6×His tag at the N-terminus linked to the ENLYFQG sequence recognized by TEV protease and a pentaglycine. The modified fluorescent protein genes were prepared in the laboratory and cloned into the pET28 plasmid, while the pET28 plasmid containing the nanobodies sequence was obtained from a commercial vendor, BioCat GmbH. E. coli strain BL21 (DE3) cells were transformed with the plasmid and precultured in LB medium at 37° C. until the OD600 value reached to ˜0.6 at which point protein expression was induced by addition of IPTG to a final concentration of 0.3 mM, followed by further cell culture at 25° C. overnight. After cell lysis by sonication, these proteins were purified by Ni-NTA affinity chromatography and size-exclusion chromatography using a Superdex 75 increase 10/300 column (GE Healthcare).

### Sortase-Mediated Modification and Cage Characterization

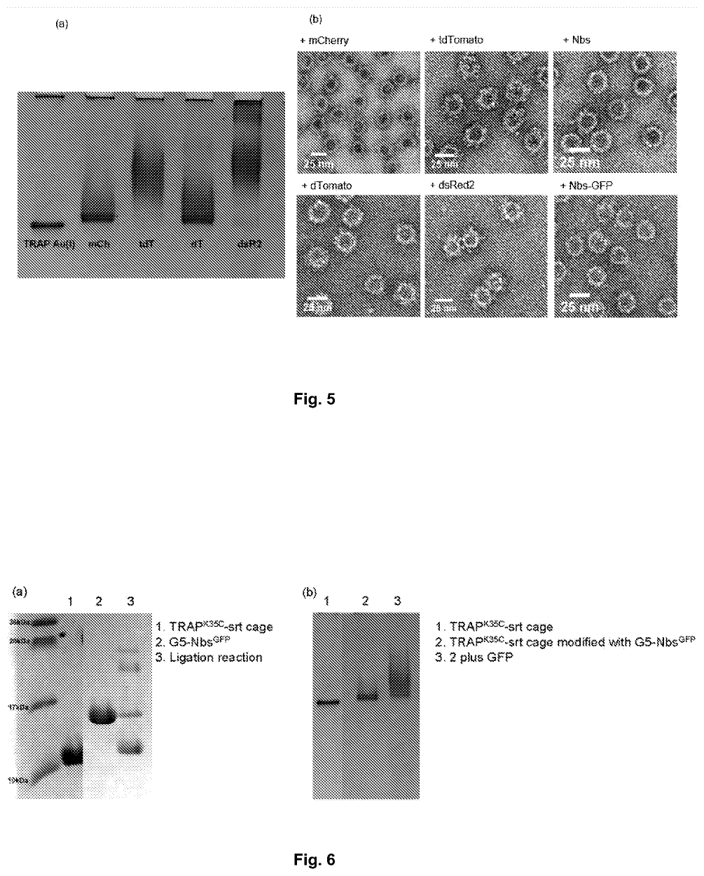

Conjugation of the TRAP cages with fluorescent proteins was performed in a PBS buffer. Proteins were mixed in the reaction buffer to final concentration of 40 μM TRAP with respect to monomer, 10 μM fluorescent proteins, and 3 μM sortase A (SrtA). The reaction was carried out for 2 hours at room temperature. Part of the reaction mixtures were analyzed by native-PAGE (FIG. 5a). The resulting cages were then purified by size-exclusion chromatography using a Superose6 increase 10/300 column (GE Healthcare) in 2×PBS buffer. Isolated cages were subsequently analyzed by negative-stain transmission electron microscope (TEM) (FIG. 5b) and dynamic light scattering (DLS). For TEM, the protein samples were diluted to 0.04 mg/mL. Copper grids (FC4000u100, Lab Soft) were glow-discharged (Leica EM Ace200, Leica Microsystems), and then 4 μL of the protein samples were applied to them and left for 1 minute. The grids were then dried using a filter paper, and 4 uL of 2% uranyl acetate was applied to the grids, and dried immediately with the same method. Then, 4 uL of 2% uranyl acetate were transferred to the grid for 15 s, and dried. Samples were then visualized on a JOEL-1230 electron microscope with 80 kV operation. The DLS measurement was performed on a Malvern ZetaSizer Nano S.

An analogous protocol was used for decoration with nanobodies. For the binding of Nbs^{GFP}, GFP was mixed in PBS with the modified TRAP cages, to a final concentration of 13 μM of TRAP and 2 μM of GFP, and kept at room temperature for 30 minutes. The resulting reaction mixtures were analyzed by SDS- and native-PAGE (FIG. 6).

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps ofany method or process so disclosed.

1. An artificial TRAP-cage comprising a selected number of TRAP rings and a plurality of external decorations attached thereto.

2. The cage according to claim 1, wherein the external decorations are selected from the group comprising nanobodies, antibodies, epitopes, antigens, proteins, peptides, cell penetrating peptides, antigenic peptides, polypeptides, nucleic acids, signaling molecules, lipids, oligosaccharides, dye molecules, inorganic nanoparticles, specific ligands and small molecule therapeutics or fragments thereof.

3. The cage according to either claim 1 or 2, wherein the external decoration is a viral, microbial or cancer antigen.

4. The cage according to any preceding, wherein the external decorations are the same or different from one another.

5. The cage according to any preceding claim, wherein at least one of the external decorations comprises a cell penetrating agent to promote intracellular delivery of the cage.

6. The cage according to claim 5, wherein the cell penetrating agent is PTD4.

7. The cage according to any preceding claim, wherein the external surface of the TRAP-cage is modified to attach the external decoration by one or more of:
(i) chemical modification;
(ii) enzymatic coupling;
(iii) bio-conjugation;
(iv) genetic coupling; and
(v) click chemistry.

8. The cage according to any preceding claim, wherein the external decoration is attached to an externally facing cysteine residue of the TRAP-cage.

9. The cage according to any preceding claim, wherein the attachment comprises chemical modification of the cysteine residue, preferably, the chemical modification is by cysteine, maleimide-based conjugation.

10. The cage according to any preceding claim, wherein the chemical modification comprises lysine amide-based conjugation.

11. The cage according to any preceding claim, where the attachment comprises enzymatic coupling, preferably by a peptide ligase.

12. The cage according to claim 11, wherein the peptide ligase is a sortase, asparaginyl endoprotease, trypsin related enzyme or a subtilisin-derived variant.

13. The cage according to any preceding claim, wherein the attachment comprises bio-conjugation, preferably maleimide labelled fluorescent dyes for attachment of surface thiols.

14. The cage according to any preceding claim, wherein the bio-conjugation comprises an azide-reactive side chain, preferably the azide-reactive side chain is DBCO.

15. The cage according to any preceding claim, wherein the genetic coupling comprises fusion to a C-terminus of TRAP.

16. The cage according to any preceding claim, where the N-terminus sequence of the external decoration is fused to a C-terminus sequence of TRAP that is available on the exterior of the TRAP-cage.

17. The cage according to any preceding claim, wherein the external decoration is conjugated using SpyCatcher/SpyTag conjugation.

18. The cage according to any preceding claim that further includes an internal cargo encapsulated therein.

19. The cage according to any preceding claim wherein the number of TRAP rings in the TRAP-cage is between 6 to 60.

20. The cage according to claim 19 wherein the number of TRAP rings in the TRAP-cage is 12, 20 or 24, preferably 24.

21. The cage according to any preceding claim, wherein the TRAP rings are held in place by cross-linkers which comprises one or more programmable cross-linkers.

22. The cage according to any preceding claim, wherein the artificial TRAP-cage protein is modified to comprise any one or more of the following mutations selected from the group comprising K35C, K35H, R64S, K35C/R64S, K35H/R64S, S33C, S33H, S33C/R64S, S33H/R64S, S33C/K35H S33H/K35H, S33C/K35C and S33H/K35C.

23. Use of the artificial TRAP-cage according to any preceding claim as a delivery vehicle for delivery of its external decoration.

24. Use according to claim 23 wherein the delivery is for intracellular delivery.

25. Use of the artificial TRAP-cage according to any one of claims 1 to 22 as a vaccine.

26. Use of the cage according to any preceding claim as a medicament.

27. A method of treating a patient, comprising administering a cage according to any one of claims 1 to 22 to said patient.

28. The cage according to any one of claims 1 to 22 for use in treating a disease in a patient, preferably wherein the patent has cancer, vascular disease, cardiovascular disease, diabetes, infection, auto-immune condition, neurological/neurodegenerative disease, arthritis and respiratory disease.

30. A method of making an artificial TRAP-cage, the method comprising:
(i) obtaining TRAP ring units by expression of the TRAP ring units in a suitable expression system and purification of the said units from the expression system;
(ii) conjugation of the TRAP ring units by at least one free thiol linkage with a cross-linker;
(iii) formation of the TRAP-cage by self-assembly and modification of an external surface of the formed TRAP-cage to what is appropriate for the external decoration that is to be attached to the cage exterior surface;
(iv) decorating the external surface of the TRAP-cage with a moiety, preferably one selected from the group comprising nanobodies, antibodies, epitopes, antigens, proteins, peptides, cell penetrating peptides, antigenic peptides, polypeptides, nucleic acids, signaling molecules, lipids, oligosaccharides, dye molecules, inorganic nanoparticles, specific ligands and small molecule therapeutics or fragments thereof; and
(v) purification and isolation of the TRAP-cages.

31. The method of claim 30, wherein step (i) the expression system is from a cell-based expression system or other expression systems such as cell-free or plant expression systems.

32. The method of either of claims 30 or 31, wherein purification of the said units from the expression system of step (i) by using FPLC-based purification employing appropriate columns such as a mixture of affinity based and size exclusion columns.

33. The method of any of claims 30 to 32, wherein the modification of step (iii) is selected from the group comprising:
(i) chemical modification;
(ii) enzymatic coupling;
(iii) bio-conjugation;
(iv) genetic coupling; and
(v) click chemistry.

34. The method according to any one of claims 30 to 33, wherein the external decoration is attached to an externally facing cysteine residue of the TRAP-cage.

35. The method according to any one of claims 30 to 34, wherein the attachment comprises chemical modification of the cysteine residue, preferably, the chemical modification is by cysteine, maleimide-based conjugation.

36. The method according to any one of claims 30 to 35, wherein the chemical modification comprises lysine amide-based conjugation.

37. The method according to any one of claims 30 to 36, where the attachment comprises enzymatic coupling, preferably by a peptide ligase.

38. The method according to claim 37, wherein the peptide ligase is a sortase, asparaginyl endoprotease, trypsin related enzyme or a subtilisin-derived variant.

39. The method according to any one of claims 30 to 38, wherein the attachment comprises bio-conjugation, preferably maleimide labelled fluorescent dyes for attachment of surface thiols.

40. The method according to any one of claims 30 to 39, wherein the bio-conjugation comprises an azide-reactive side chain, preferably the azide-reactive side chain is DBCO.

41. The method according to any one of claims 30 to 40, wherein the genetic coupling comprises fusion to a C-terminus of TRAP.

42. The method according to any one of claims 30 to 41, where the N-terminus sequence of the external decoration is fused to a C-terminus sequence of a TRAP protein on the exterior of the TRAP-cage.

43. The method according to any one of claims 30 to 42, wherein the method comprises SpyCatcher/SpyTag conjugation of the external decoration to an exterior surface of the TRAP-cage

44. A TRAP-cage produced by the method of any one of claims 30 to 43.

45. A method of treatment of an individual in need of therapy suffering from a condition selected from the group comprising cancer, vascular disease, cardiovascular disease, diabetes, infection, cellular senescence, auto-immune condition, neurological/neurodegenerative disease, arthritis and respiratory disease, the method comprising administering a therapeutically effective amount of an artificial TRAP-cage bearing one or more external decorations selected from the group comprising nanobodies, antibodies, epitopes, antigens, proteins, peptides, cell penetrating peptides, antigenic peptides, polypeptides, nucleic acids, signaling molecules, lipids, oligosaccharides, dye molecules, inorganic nanoparticles, specific ligands and small molecule therapeutics or fragments thereof.

46. A method of vaccinating an individual suffering from a condition selected from the group comprising cancer, vascular disease, cardiovascular disease, diabetes, infection, cellular senescence, auto-immune condition, neurological/neurodegenerative disease, arthritis and respiratory disease, the method comprising administering a therapeutically effective amount of an artificial TRAP-cage bearing one or more external decorations selected from the group comprising nanobodies, antibodies, epitopes, antigens, proteins, peptides, cell penetrating peptides, antigenic peptides, polypeptides, nucleic acids, signaling molecules, lipids, oligosaccharides, dye molecules, inorganic nanoparticles, specific ligands and small molecule therapeutics or fragments thereof.

47. The methods of either claims 45 or 46 wherein the TRAP-cage therapeutic is administered via intranasal inhalation or injection.
